(19) United States
(12) Patent Application Publication
(10) Pub. No.: US 2026/0193608 A1
(43) Pub. Date: Jul. 9, 2026

(54) METHOD FOR THE PRODUCTION OF GAMMA DELTA T CELLS FOR IMMUNOTHERAPY APPLICATIONS

(71) Applicant: JI YAN BIOMEDICAL CO., LTD., Taipei City (TW)

(72) Inventors: Bo-Xiang ZHANG, Taipei City (TW); Hsieh-Tsung SHEN, Taipei City (TW)

(21) Appl. No.: 19/442,399

(22) Filed: Jan. 7, 2026

Related U.S. Application Data
(60) Provisional application No. 63/742,633, filed on Jan. 7, 2025.

(51) Int. Cl. C12N 5/0783; A61K 9/00; A61K 35/17; A61K 39/00; A61K 40/11; A61P 35/00; C12N 5/00

(52) U.S. Cl. CPC C12N 5/0636; A61K 9/0019; A61K 35/17; A61K 39/00; A61K 40/11; A61P 35/00; C12N 5/0087; A61K 2039/5158; C12N 2500/32; C12N 2500/46; C12N 2500/90; C12N 2501/10; C12N 2501/2302; C12N 2501/2305; C12N 2501/2315

(57) ABSTRACT

The present disclosure relates to a field of immunotherapy. Particularly, the present disclosure provides methods for production of gamma delta T (γδ T) cells and the γδ T cells produced therefrom.

### PRIORITY INFORMATION

This application claims benefit of and priority to U.S. Provisional Patent Application No. 63/742,633, filed Jan. 7, 2025, the contents of which is incorporated by reference in its entirety.

### FIELD OF THE INVENTION

The present disclosure relates to the field of immunotherapy. In Particular, the present disclosure provides methods for producing gamma delta T (γδ T) cells and the γδ T cells produced therefrom.

### BACKGROUND OF THE INVENTION

γδ T cells represent a distinct subset of T lymphocytes that present a promising avenue for T cell-based immunotherapy. Their capacity to produce significant amounts of cytokines with anti-tumor properties, along with characteristics of both innate and adaptive immune cells, makes them particularly appealing. Clinical trials utilizing a combination of in vivo and ex vivo stimulation of γδ T cells have often depended on agonists like aminobisphosphonates (n-BPs), which are associated with limited bioavailability and potential off-target toxicity (La-Beck N M, et al., Repurposing amino-bisphosphonates by liposome formulation for a new role in cancer treatment. Semin Cancer Biol 2021; 68:175-185). Phosphoantigens (pAgs) can stimulate γδ T cell populations more effectively than conventional nitrogen-containing bisphosphonates (n-BPs) (Raverdeau M, Cunningham S P, Harmon C, Lynch L. γδ T cells in cancer: a small population of lymphocytes with big implications. Clin Transl Immunology 2019; 8: e01080). Nonetheless, the clinical effectiveness of γδ T-cell immunotherapies is constrained by challenges related to the pharmacodynamic and pharmacokinetic profiles of frequently used agonists, the difficulty of infused T cells in penetrating solid tumors, and a limited understanding of γδ T-cell checkpoint inhibition.

Therefore, there is need to develop new therapeutics for improving γδ T-cell immunotherapies.

### SUMMARY OF THE INVENTION

The present disclosure relates to a novel method for the production of Vδ²⁺ γδ T cells for therapeutic applications. The method includes isolation, expansion, and activation of Vδ²⁺ γδ T cells from peripheral blood mononuclear cells under optimized conditions.

In one aspect, the present disclosure provides a method for producing a cell population comprising enriched Vδ²⁺ γδ T cells, comprising:
(a) providing peripheral blood mononuclear cells (PBMCs); optionally the Vδ²⁺ γδ T cells contained in the PBMCs are separated by immunomagnetic separation to obtain a Vδ²⁺ γδ T cell subpopulation;
(b) culturing the PBMCs or Vδ²⁺ γδ T cell subpopulation in a medium containing zoledronic acid and one or more growth factors having interleukin-like activity to specifically enrich Vδ²⁺ γδ T cells to obtain an enriched Vδ²⁺ γδ T cell population; and
(c) harvesting the expanded Vδ²⁺ γδ T cell population.

In a particular embodiment, the present disclosure provides a method for producing a cell population comprising enriched Vδ²⁺ γδ T cells, comprising:
(a1) providing peripheral blood mononuclear cells (PBMCs);
(b1) culturing the PBMCs in a medium containing zoledronic acid and one or more growth factors having interleukin-like activity to specifically enrich Vδ²⁺ γδ T cells to obtain an enriched Vδ²⁺ γδ T cell population; and
(c1) harvesting the expanded Vδ²⁺ γδ T cell population; or
(a2) providing peripheral blood mononuclear cells (PBMCs);
(i) separating Vδ2⁺ γδ T cells by immunomagnetic separation to obtain a Vδ2⁺ γδ T cell subpopulation;
(b2) culturing the PBMCs in a medium containing zoledronic acid and one or more growth factors having interleukin-like activity to specifically enrich Vδ²⁺ γδ T cells to obtain an enriched Vδ²⁺ γδ T cell population; and
(c2) harvesting the expanded Vδ²⁺ γδ T cell population; or
In one embodiment, after step (b), (b1) or (b2), the method further comprises a step (e) of separating the enriched Vδ²⁺ γδ T cell population by immunomagnetic separation to remove non-Vδ²⁺ γδ T cells.

In one embodiment, after step (b), (b1), (b2) or (e), the method further comprises a step (f) of expanding the enriched Vδ²⁺ γδ T cell population by culturing it for 7-28 days to obtain the expanded Vδ²⁺ γδ T cell population.

In one embodiment, the Vδ²⁺ γδ cells in the Vδ²⁺ γδ T cell population are unmodified.

In some embodiments, the PBMCs are obtained from a peripheral blood, umbilical cord blood, or fractions thereof.

In one embodiment, the zoledronic acid is at a concentration of about 1 to about 30 μM, about 1 to about 28 μM, about 1 to about 25 μM, about 1 to about 23 μM, about 1 to about 20 μM, about 1 to about 18 μM, about 1 to about 15 μM, about 1 to about 13 μM, about 1 to about 10 μM, about 1 to about 8 μM, about 1 to about 5 μM, about 3 to about 30 μM, about 3 to about 28 μM, about 3 to about 25 μM, about 3 to about 23 μM, about 3 to about 20 μM, about 3 to about 18 μM, about 3 to about 15 μM, about 3 to about 13 μM, about 3 to about 10 μM, about 3 to about 8 μM, about 3 to about 5 μM, about 5 to about 30 μM, about 5 to about 28 μM, about 5 to about 25 μM, about 5 to about 23 μM, about 5 to about 20 μM, about 5 to about 18 μM, about 5 to about 15 μM, about 5 to about 13 μM, about 5 to about 10 μM, about 8 to about 30 μM, about 8 to about 28 μM, about 8 to about 25 μM, about 8 to about 23 μM, about 8 to about 20 μM, about 8 to about 15 μM, about 8 to about 12 μM, about 8 to about 10 μM, about 10 to about 30 μM, about 10 to about 28 μM, about 10 to about 25 μM, about 10 to about 23 μM, about 10 to about 20 μM, about 10 to about 18 μM, or about 10 to about 15 μM.

In one embodiment, the medium is free of serum.

In one embodiment, the growth factor having interleukin-like activity is an interleukin.

In one embodiment, the concentration of the growth factor having interleukin-like activity ranges from about 5 ng/ml to about 200 ng/ml or about 50 IU/mL to about 1,500 IU/mL.

In one embodiment, the concentration of the growth factor having interleukin-like activity ranges from about 5 ng/ml to about 200 ng/ml, about 5 ng/ml to about 180 ng/ml, about 5 ng/ml to about 150 ng/ml, about 5 ng/ml to about 100 ng/ml, about 5 ng/ml to about 80 ng/mL, about 5 ng/ml to about 70 ng/mL, about 5 ng/ml to about 60 ng/ml, about 5 ng/ml to about 50 ng/ml, about 5 ng/ml to about 40 ng/ml, about 5 ng/ml to about 30 ng/ml, about 5 ng/mL to about 20 ng/ml, about 10 ng/ml to about 200 ng/ml, about 10 ng/ml to about 180 ng/ml, about 10 ng/ml to about 150 ng/mL, about 10 ng/ml to about 100 ng/mL, about 10 ng/mL to about 80 ng/mL, about 10 ng/ml to about 70 ng/ml, about 10 ng/ml to about 60 ng/ml, about 10 ng/mL to about 50 ng/ml, about 10 ng/mL to about 40 ng/mL, about 10 ng/ml to about 30 ng/ml, about 20 ng/ml to about 200 ng/ml, about 20 ng/ml to about 180 ng/mL, about 20 ng/mL to about 150 ng/ml, about 20 ng/ml to about 100 ng/ml, about 20 ng/ml to about 80 ng/ml, about 20 ng/ml to about 70 ng/mL, about 20 ng/ml to about 60 ng/ml, about 20 ng/ml to about 50 ng/ml, about 20 ng/ml to about 40 ng/mL, about 20 ng/ml to about 30 ng/mL, about 30 ng/ml to about 200 ng/ml, about 30 ng/mL to about 180 ng/ml, about 30 ng/mL to about 150 ng/ml, about 30 ng/mL to about 100 ng/ml, about 30 ng/mL to about 80 ng/ml, about 30 ng/ml to about 70 ng/ml, about 30 ng/ml to about 60 ng/mL, about 30 ng/ml to about 50 ng/ml or about 30 ng/ml to about 40 ng/mL.

In one embodiment, the concentration of the growth factor having interleukin-like activity ranges from about 50 IU/mL to about 1,500 IU/mL, about 50 IU/mL to about 1,400 IU/mL, about 50 IU/mL to about 1,300 IU/mL, about 50 IU/mL to about 1,200 IU/mL, about 50 IU/mL to about 1,100 IU/mL, about 50 IU/mL to about 1,000 IU/mL, about 70 IU/mL to about 1,500 IU/mL, about 70 IU/mL to about 1,400 IU/mL, about 70 IU/mL to about 1,300 IU/mL, about 70 IU/mL to about 1,200 IU/mL, about 70 IU/mL to about 1,100 IU/mL, about 70 IU/mL to about 1,000 IU/mL, 80 IU/mL to about 1,500 IU/mL, about 80 IU/mL to about 1,400 IU/mL, about 80 IU/mL to about 1,300 IU/mL, about 80 IU/mL to about 1,200 IU/mL, about 80 IU/mL to about 1,100 IU/mL, about 80 IU/mL to about 1,000 IU/mL, about 90 IU/mL to about 1,500 IU/mL, about 90 IU/mL to about 1,400 IU/mL, about 90 IU/mL to about 1,300 IU/mL, about 90 IU/mL to about 1,200 IU/mL, about 90 IU/mL to about 1,100 IU/mL, about 90 IU/mL to about 1,000 IU/mL, about 100 IU/mL to about 1,500 IU/mL, about 100 IU/mL to about 1,400 IU/mL, about 100 IU/mL to about 1,300 IU/mL, about 100 IU/mL to about 1,200 IU/mL, about 100 IU/mL to about 1,100 IU/mL or about 100 IU/mL to about 1,000 IU/mL.

In some embodiments, the interleukin is IL-2, IL-4, IL-7, IL-15, IL-21, interleukin-1β or any combination thereof. In one further embodiment, the interleukin is IL-2, IL-15, or a combination of IL-2 and IL-5. In one further embodiment, the interleukin is IL-2 in a concentration of about 100 IU/mL to about 1,000 IU/mL. In one further embodiment, the interleukin is IL-15 in a concentration of about 10 ng/ml to about 100 ng/mL. In one further embodiment, the interleukin is about 100 IU/mL to about 1,000 IU/mL of IL-2 in combination with about 10 ng/ml to about 100 ng/ml of IL-15.

In one embodiment, the medium containing IL-15 maintains a constant expansion of Vδ²⁺ γδ T-cells at a high level over time.

In one embodiment, the zoledronic acid mediated Vδ²⁺ γδ T-cell expansion can be increased by IL-15.

In one embodiment, the medium further comprises synthetic growth factors and amino acids tailored for T cell expansion. Examples of growth factor include, but are not limited to, neurotrophic factor (BDNF), epidermal growth factor (EGF), fibroblast growth factor (FGF), insulin-like growth factor (IGF-1), erythropoietin (EPO), vascular growth factor (VEGF), transforming growth factor beta (TGF-β), nerve growth factor (NGF) and platelet derived growth factors (PDGF).

Examples of amino acid include, but are not limited to, glutamine (Gln), alanine (Ala), serine (Ser), leucine (Leu), methionine (Met), arginine (Arg), cysteine (Cys) and cystine (Cys-Cys).

In one embodiment, the step of expanding the Vδ²⁺ γδ T cells is conducted at 37° C. and 5% CO₂.

In one embodiment, the harvested Vδ²⁺ γδ T cells are cryopreserved in a solution containing DMSO and a protective cryoprotectant.

In one aspect, the present disclosure provides a cell population produced from the method described herein.

In one embodiment, the present disclosure provides a Vδ²⁺ γδ T cell population, comprising enriched Vδ²⁺ γδ T cells.

In some embodiments, the Vδ²⁺ γδ T cell population described herein comprises at least about 85%, 88%, 90%, 91%, 92%, 93%, 94%, 95%, 96%, 97%, 98% or 99% Vδ²⁺ γδ T cells with over about 70%, 75%, 80%, 85%, 90%, 91%, 92%, 93%, 94%, 95%, 96%, 97%, 98% or 99% post-expansion viability.

In one embodiment, the Vδ²⁺ γδ T cell population described herein is of therapeutic grade.

In one embodiment, the Vδ²⁺ γδ T cell population described herein has low residual αβ T cells. In some embodiments, the enriched γδ T cell population has residual αβ T cell less than about 0.5%, 1%, 2%, 3%, 4%, 5%, 6%, 7%, 8%, 9% or 10%.

In one embodiment, the Vδ²⁺ γδ T cell population described herein comprises at least about 95%, 96%, 97%, 98% or 99% Vδ²⁺ γδ T cells with over about 90%, 91%, 92%, 93%, 94%, 95%, 96%, 97%, 98% or 99% post-expansion viability.

In some embodiments, the Vδ²⁺ γδ T cells described herein express one or more CD3, CD45 and NKG2D in an intensity higher than about 70%, 75%, 80%, 85%, 90%, 95%, 96%, 97%, 98%, 98.5%, 99% or 99.5%.

In some embodiments, the Vδ²⁺ γδ T cells described herein express NKG2D in an intensity higher than about 95%, 96%, 97%, 98%, 98.5%, 99% or 99.5%. In a further embodiment, the Vδ²⁺ γδ T cells described herein express NKG2D in an intensity higher than about 98%.

In some embodiments, the Vδ²⁺ γδ T cells described herein express CD3 in an intensity higher than about 95%, 96%, 97%, 98%, 98.5%, 99% or 99.5%. In a further embodiment, the Vδ²⁺ γδ T cells described herein express CD3 in an intensity higher than about 99%.

In some embodiments, the Vδ²⁺ γδ T cells described herein express CD45 in an intensity higher than about 95%, 96%, 97%, 98%, 98.5%, 99% or 99.5%. In a further embodiment, the Vδ²⁺ γδ T cells described herein express CD45 in an intensity higher than about 98%.

In some embodiments, the Vδ²⁺ γδ T cells described herein express CD3, CD45 and NKG2D in an intensity higher than about 70%, 75%, 80%, 85%, 90%, 95%, 96%, 97%, 98%, 98.5%, 99% or 99.5%. In a further embodiment, the Vδ²⁺ γδ T cells described herein express CD3, CD45 and NKG2D in an intensity higher than about 95%, 96%, 97%, 98%, 98.5%, 99% or 99.5%.

In another aspect, the present disclosure provides a method of modulating an immune response in a subject, comprising administering the enriched Vδ²⁺ γδ T cell population described herein to the subject.

In another aspect, the present disclosure provides a method for use in immunotherapy in a subject, comprising administering the enriched Vδ²⁺ γδ T cell population described herein to the subject.

In some embodiments, the immunotherapy provides multifunctional immune modulation.

In some embodiments, the immunotherapy provides potent effector functionality and cytokine-producing capability of the expanded Vδ²⁺ γδ T cells upon tumor cell recognition.

In some embodiments, the immunotherapy is chimeric antigen receptor (CAR) T cell therapy.

In another aspect, the present disclosure provides a method for treating a cancer in a subject, comprising administering the enriched Vδ²⁺ γδ T cell population described herein to the subject.

In some embodiments, the cancer is sarcoma, basal cell skin cancer, lymphoma, leukemia, lymphoproliferative disorder, plasmacytoma, histiocytoma, adenoma, carcinomas of solid tissues, hypoxic tumor, genitourinary cancer, hematopoietic cancer, nervous system cancer, bile duct cancer, cervical cancer, squamous cell cancer, endometrial cancer, esophageal cancer, head and neck cancer, glioblastoma (GBM) (such as glioblastoma multiforme), kidney cancer, hepatocellular carcinoma, liver cancer, lung cancer, pancreatic cancer, melanoma, Merkel cell cancer, mesothelioma, stomach cancer, breast cancer or triple-negative breast cancer. In further embodiments, the cancer is breast cancer, lung cancer, hepatocellular carcinoma, liver cancer, pancreatic cancer or GBM.

In some further embodiments, the leukemia is chronic lymphocytic leukemia, chronic myelogenous leukemia, acute myelogenous leukemia, acute lymphoblastic leukemia, or T cell and B cell leukemias; the lymphoma is Hodgkin's lymphorma or non-Hodgkin lymphoma; and the genitourinary cancer is cervical cancer or bladder cancer.

In a further aspect, the present disclosure provides a method for treating an infectious disease in a subject, comprising administering the enriched Vδ²⁺ γδ T cell population described herein to the subject. In some embodiment, the infectious disease is bacterial infections such as those caused by mycobacteria (e.g. tuberculosis), viral infections such as those caused by herpes simplex viruses (HSV), human immunodeficiency viruses (HIV), the hepatitis viruses, SARS-CoV-2 viruses, papillomaviruses (HPV), Epstein-Barr viruses (EBV), measles viruses, cytomegaloviruses (CMV), hepatitis C viruses (HCV), or influenza viruses, and parasitic infections such as those caused by plasmodium (e.g. malaria).

In one embodiment, the infectious disease is a viral infection. In some embodiments, the viral infection is caused by human immunodeficiency virus (HIV), SARS-CoV-2, papillomavirus (HPV), herpes simplex virus (HSV), Epstein-Barr virus (EBV), measles virus, cytomegalovirus (CMV), hepatitis C virus (HCV), or influenza virus.

In a further aspect, the present disclosure provides a method for vaccinating an animal comprising administering the enriched Vδ²⁺ γδ T cell population described herein to the animal.

### BRIEF DESCRIPTION OF THE DRAWINGS

FIG. 1 shows that zoledronic acid mediates Vδ²⁺ γδ T-cell expansion at different IL-2 concentrations depending on the concentrations of zoledronic acid.

FIG. 2 shows the effect of different cytokine combinations on zoledronic acid mediated γδ T-cell expansion.

FIG. 3 shows that zoledronic acid mediates Vδ²⁺ γδ T-cell expansion at different concentrations of IL-15.

FIG. 4 shows constant long-term Vδ²⁺ γδ T-cell expansion by adding IL-15.

FIGS. 5 (A) to (E) show flow cytometric characterization of expanded Vδ2⁺ γδ T cells. (A) Live-cell gating of cultured PBMC-derived cells. Peripheral blood mononuclear cells (PBMCs) were isolated from healthy donors and subjected to Vδ²⁺ γδ T-cell expansion. Zombie dye staining identified 95.65% of cells as viable, confirming high survival during the 14-day culture period. (B) Identification of CD45⁺CD³⁺ T-cell gate. Analysis of live cells showed that 98.59% of the population consisted of CD45⁺CD³⁺ lymphocytes, demonstrating successful enrichment of T-lineage cells following culture. (C) Quantification of residual αβ T cells. Within the CD³⁺ fraction, TCRαβ cells accounted for 99.90%, with only 0.09% of the population expressing TCRαβ⁺, confirming minimal residual αβ T-cell contamination after the expansion process. (D) Enrichment of Vδ²⁺ γδ T cells. Among TCRαβ cells, Vδ²⁺ γδ T cells constituted 96.11% of the population, whereas Vδ¹⁺ cells were undetectable (0.00%), indicating highly selective expansion of the Vδ² subset. (E) Surface expression of NKG2D in expanded Vδ²⁺ γδ T cells. Expanded Vδ²⁺ γδ T cells exhibited high NKG2D expression (98.30%), indicating acquisition of an activated cytotoxic phenotype suitable for anti-tumor applications.

FIGS. 6 (A) to (C) show cytotoxic activity of expanded Vδ²⁺ γδ T cells against multiple human cancer cell lines . . . (A) Cytotoxicity against MDA-MB231 human breast cancer cells. Expanded Vδ²⁺ γδ T cells were co-cultured with MDA-MB231 cells at effector-to-target (E/T) ratios of 0:1, 1:1, 2:1, 5:1, and 10:1 for 24 hours. Annexin V/PI staining demonstrated a dose-dependent increase in tumor cell apoptosis, with >80% apoptosis observed at an E/T ratio of 5:1 and sustained at 10:1. (B) Cytotoxicity against Huh-7 human liver cancer cells. Expanded Vδ²⁺ γδ T cells induced apoptosis in Huh-7 cells in an E/T ratio-dependent manner. At 5:1 and 10:1 ratios, >80% of tumor cells were apoptotic. Limited cell recovery at higher ratios was noted due to extensive cell death and fragmentation during co-culture. (C) Cytotoxicity against A549 human lung cancer cells. A549 tumor cells exhibited a higher resistance to Vδ²⁺ γδ T-cell-mediated cytotoxicity. Dose-dependent apoptosis was observed, with >80% apoptosis achieved at the 10:1 E/T ratio, indicating cell line-specific differences in susceptibility to Vδ²⁺ γδ T-cell killing.

FIGS. 7 (A) to (D) show the cytokine secretion profile of unmodified Vδ²⁺ γδ T cells following co-culture with tumor target cell. (A) IFN-γ secretion at increasing effector-to-target ratios. Unmodified Vδ²⁺ γδ T cells were co-cultured with tumor cells at E:T ratios of 0:1, 0.5:1, 1:1, and 2:1 for 24 hours. ELISA quantification demonstrated a dose-dependent increase in IFN-γ secretion, reaching levels approaching 300 μg/mL at the 2:1 ratio, indicating strong Th1-type activation. (B) IL-4 secretion at increasing effector-to-target ratios. IL-4 secretion progressively increased with higher E:T ratios, showing that Vδ²⁺ γδ T cells retain the capacity to produce Th2-associated cytokines following tumor cell stimulation. (C) IL-9 secretion at increasing effector-to-target ratios. IL-9 production exhibited a dose-dependent increase, with concentrations approaching 100 μg/mL at the 2:1 E:T ratio, indicating activation of additional effector cytokine pathways. (D) TNF-α secretion at increasing effector-to-target ratios. TNF-α levels increased in a ratio-dependent manner, almost reaching 40 μg/mL at the 2:1 ratio, consistent with a strong pro-inflammatory and cytotoxic activation profile.

FIGS. 8 (A) and (B) show in vivo anti-tumor efficacy of unmodified γδ T cells in a Huh7 hepatocellular carcinoma xenograft model. (A) Dose-dependent tumor growth inhibition following co-inoculation of Vδ²⁺ γδ T cells with Huh7 cells. (B) Therapeutic efficacy of systemically administered γδ T cells in established tumors.

FIG. 9 shows in vivo anti-tumor efficacy of unmodified γδ T cells in an A549 lung cancer xenograft model. Unmodified Vδ²⁺ γδ T cells markedly inhibited tumor growth in the co-injection model.

FIGS. 10 (A) and (B) show cytotoxic activity of unmodified Vδ²⁺ γδ T cells against BxPC-3 pancreatic cancer cells measured by real-time cell analysis. (A) Real-time cell index profiles of BxPC-3 cells cultured alone or co-cultured with unmodified Vδ²⁺ γδ T cells at BxPC-3:T ratios of 1:2, 1:3, and 1:4. (B) Quantitative comparison of cell indices at 24 and 48 hours after γδ T cell addition confirmed significant growth inhibition in all treatment groups, with near-complete suppression of BxPC-3 cell proliferation at ratios of 1:4.

FIGS. 11 (A) to (D) show that γδ T cells exhibit superior cytotoxic effects against GBM cells (U87MG). (A) Representative flow cytometry plots of CD³⁺/CD45⁺ T cells showing the proportion of αβ T cells (TCRαβ⁺) and γδ T cells (TCRγδ⁺). (B) γδ T cells exhibit a distinct TCRγδ⁺ population, whereas αβ T cells predominantly express TCRαβ⁺. (C) Quantification of viable GBM cells by luciferase assay after co-culture with αβ or γδ T cells at 1:1 and 1:0.5 GBM:T cell ratios. (D) Cytotoxicity of γδ T cells and αβ T cells against GBM cells. Statistical significance: *p<0.001, p<0.05, n.s=not significant.

FIGS. 12 (A) to (D) show in vivo anti-tumor efficacy of unmodified γδ T cells in an orthotopic glioblastoma (U87MG) mouse model. (A, B) Baseline tumor bioluminescence before Vδ²⁺ γδ T-cell administration (Day 0). An orthotopic glioblastoma model was generated by stereotactic implantation of U87MG-luc cells into the right striatum of immunodeficient mice. Bioluminescence imaging (BLI) was performed on Day 0 to confirm tumor engraftment and establish baseline tumor burden. (C, D) Tumor bioluminescence following intracranial Vδ²⁺ γδ T-cell administration (Day 2). Unmodified Vδ²⁺ γδ T cells were injected intracranially at the tumor site on Day 0, as indicated by the injection marker. BLI analysis on Day 2 demonstrated a substantial reduction in luminescence intensity relative to baseline, indicating early in vivo anti-tumor activity of the administered Vδ²⁺ γδ T cells.

FIGS. 13 (A) to (C) show inhibition of glioblastoma growth by co-injection of unmodified Vδ²⁺ γδ T cells in an orthotopic U87MG mouse model. (A) Representative bioluminescence imaging (BLI) of mice implanted orthotopically with U87MG-luc cells alone (U87L) or co-injected with unmodified Vδ²⁺ γδ T cells at U87:T ratios of 1:2, 1:4, and 1:8. (B) Quantitative BLI analysis showed significant reduction in photon flux in the GDT-treated groups compared with the control group (U87L). (C) Kaplan-Meier survival analysis demonstrated prolonged survival in mice receiving Vδ²⁺ γδ T cells, with complete tumor growth suppression in the 1:4 and 1:8 groups.

### DETAILED DESCRIPTION OF THE INVENTION

When items are connected by the conjunction “and,” it should not be interpreted as necessitating the presence of each individual item in the group; instead, it should be understood as “and/or,” unless explicitly stated otherwise. Likewise, when items are linked by “or,” it should not be construed as requiring that the items be mutually exclusive; rather, it should also be interpreted as “and/or,” unless explicitly stated otherwise. Additionally, while items, elements, or components of the invention may be referred to in the singular form, the plural form is considered to be included within the scope unless there is a clear indication limiting it to the singular.

As used herein, the terms “a,” “an,” and “the” should be interpreted to encompass both singular and plural forms, unless specified otherwise. Therefore, “a,” “an,” and “the” (along with their grammatical variations, as applicable) refer to one or more entities.

The term “pharmaceutically acceptable” as used in this context refers to compounds, materials, compositions, and/or dosage forms that, according to sound medical judgment, are appropriate for contact with the tissues of a subject (whether human or non-human animal) without causing excessive toxicity, irritation, allergic reactions, or other complications, while maintaining a reasonable benefit/risk ratio. Additionally, each carrier, excipient, and similar component must be deemed “acceptable” in terms of compatibility with the other ingredients in the formulation. Suitable carriers and excipients can be identified in standard pharmaceutical references.

The term “treatment” is understood as meaning to lessen or decrease at least one sign, symptom, indication, or effect of a specific disease or condition. As used herein, “prevention” is understood to mean limiting, reducing the rate or degree of onset, or inhibiting the development of at least one sign or symptom of a disease or condition.

As used herein, the term “subject” is any animal that can benefit from the administration of a compound or composition as disclosed herein. In some embodiments, the subject is a mammal, for example, a human, a primate, a dog, a cat, a horse, a cow, a pig, a rodent, such as a rat or mouse. Typically, the mammal is a human.

The term “effective amount” as referenced here refers to the quantity of the cells that is sufficient to positively impact the condition being treated, while remaining low enough to minimize the risk of serious side effects, all within the bounds of prudent medical judgment.

The present disclosure relates to novel methods for the isolation and the selective in vitrolex vivo expansion and differentiation of γδ T cells (such as Vδ²⁺ γδ T cells), and their clinical application. Accordingly, the present disclosure provides a scalable and reproducible method for producing therapeutic-grade Vδ²⁺ γδ T cells described herein suitable for clinical immunotherapy applications. The method begins with isolating PBMCs using Ficoll-Paque density gradient centrifugation. Optionally, the isolated PBMCs are enriched for Vδ²⁺ γδ T cells using immunomagnetic beads. The selected cells are then cultured in a serum-free medium supplemented with zoledronic acid and cytokines (such as IL-2, IL-4, IL-7, IL-15, IL-21, interleukin-1β or any combination thereof) to promote cell growth and functional activation. Optionally, the enriched Vδ²⁺ γδ T cell population can be treated with immunomagnetic beads to remove the non-Vδ²⁺ γδ T cells to further enrich the Vδ²⁺ γδ T cells. The enriched Vδ²⁺ γδ T cell populations described herein cells are further expanded, harvested, washed, and cryopreserved under Good Manufacturing Practices GMP) conditions.

The PBMCs may be isolated from a sample of blood using techniques known in the art such as density gradient centrifugation. Optionally, the Vδ²⁺ γδ T cell subpopulation is selected from the isolated PBMCs using immunomagnetic separation to obtain Vδ²⁺ γδ T cell population having the selected Vδ²⁺ γδ T cell subpopulation. Immunomagnetic separation (IMS) is a tool that can efficiently isolate cells from bodily fluid or cultured cells. A mixture of cell population will be placed in a magnetic field where the cells then attach to super paramagnetic beads. Antibodies coating paramagnetic beads will bind to antigens present on the surface of cells thus capturing the cells and facilitating the concentration of these bead-attached cells.

Gamma delta (γδ) T cells are considered the archetype of unconventional T cells and constitute a relatively small fraction of T cells found in peripheral blood. They are characterized by the presence of heterodimeric T-cell receptors (TCRs) made up of γ and δ chains, distinguishing them from the more familiar CD4+ helper T cells and CD8+ cytotoxic T cells, which express αβ TCRs. The process of (thymic) selection for γδ T cells remains largely unclear. In general, γδ T cells are enriched in epithelial and mucosal tissues where they are thought to serve as the first line of defense against pathogenic challenge. The majority of γδ T cells are activated in an MHC-independent manner, in striking contrast to MHC-restricted αβ T cells. The antigens recognized by most γδ T cells are still unknown.

γδ T cells exhibit significant functional versatility upon recognizing infected or transformed cells. They produce various cytokines (such as IFN-γ, TNF-α, and IL-17) and chemokines (including RANTES, IP-10, and lymphotactin), engage in the cytolysis of these target cells through mechanisms involving performing, granzymes, and TRAIL, and interact with a range of other cell types, including epithelial cells, monocytes, dendritic cells, neutrophils, and B cells. Moreover, γδ T cells can recognize and eliminate a wide range of cancers without the need for MHC restriction, underscoring their promise for universal immunotherapy. This stands in contrast to αβ T-cell mediated immunotherapy, which is restricted by MHC.

The method of the present disclosure and the cell population produced therefrom provide an enriched Vδ²⁺ γδ T cell subpopulation. Vδ²⁺ γδ T cells form the predominant human 78 T-cell population in peripheral blood and mediate T-cell receptor (TCR)-dependent anti-microbial and anti-tumor immunity.

A medium containing zoledronic acid and one or more growth factors having interleukin-like activity is used to culture the γδ T cell population having selected Vδ²⁺ γδ T cell subpopulation to enrich the Vδ²⁺ γδ T cells. The concentration of zoledronic acid is, for example, about 1 to about 30 μM, about 1 to about 28 μM, about 1 to about 25 μM, about 1 to about 23 μM, about 1 to about 20 μM, about 1 to about 18 μM, about 1 to about 15 μM, about 1 to about 13 μM, about 1 to about 10 μM, about 1 to about 8 μM, about 1 to about 5 μM, about 3 to about 30 μM, about 3 to about 28 μM, about 3 to about 25 μM, about 3 to about 23 μM, about 3 to about 20 μM, about 3 to about 18 μM, about 3 to about 15 μM, about 3 to about 13 μM, about 3 to about 10 μM, about 3 to about 8 μM, about 3 to about 5 μM, about 5 to about 30 μM, about 5 to about 28 μM, about 5 to about 25 μM, about 5 to about 23 μM, about 5 to about 20 μM, about 5 to about 18 μM, about 5 to about 15 μM, about 5 to about 13 μM, about 5 to about 10 μM, about 8 to about 30 μM, about 8 to about 28 μM, about 8 to about 25 μM, about 8 to about 23 μM, about 8 to about 20 μM, about 8 to about 15 μM, about 8 to about 12 μM, about 8 to about 10 μM, about 10 to about 30 μM, about 10 to about 28 μM, about 10 to about 25 μM, about 10 to about 23 μM, about 10 to about 20 μM, about 10 to about 18 μM, or about 10 to about 15 μM.

The concentration of the growth factor having interleukin-like activity ranges from about 5 ng/ml to about 200 ng/ml or about 50 IU/mL to about 1,500 IU/mL. In one embodiment, the concentration of the growth factor having interleukin-like activity ranges from about 5 ng/ml to about 200 ng/mL, about 5 ng/ml to about 180 ng/ml, about 5 ng/ml to about 150 ng/ml, about 5 ng/ml to about 100 ng/ml, about 5 ng/ml to about 80 ng/ml, about 5 ng/ml to about 70 ng/mL, about 5 ng/ml to about 60 ng/ml, about 5 ng/ml to about 50 ng/ml, about 5 ng/ml to about 40 ng/mL, about 5 ng/ml to about 30 ng/ml, about 5 ng/mL to about 20 ng/mL, about 10 ng/ml to about 200 ng/ml, about 10 ng/ml to about 180 ng/ml, about 10 ng/ml to about 150 ng/ml, about 10 ng/ml to about 100 ng/ml, about 10 ng/ml to about 80 ng/mL, about 10 ng/ml to about 70 ng/ml, about 10 ng/ml to about 60 ng/ml, about 10 ng/ml to about 50 ng/ml, about 10 ng/ml to about 40 ng/ml, about 10 ng/ml to about 30 ng/ml, about 20 ng/ml to about 200 ng/ml, about 20 ng/ml to about 180 ng/mL, about 20 ng/ml to about 150 ng/mL, about 20 ng/ml to about 100 ng/mL, about 20 ng/ml to about 80 ng/mL, about 20 ng/ml to about 70 ng/ml, about 20 ng/mL to about 60 ng/ml, about 20 ng/ml to about 50 ng/mL, about 20 ng/ml to about 40 ng/ml, about 20 ng/ml to about 30 ng/ml, about 30 ng/ml to about 200 ng/ml, about 30 ng/ml to about 180 ng/ml, about 30 ng/ml to about 150 ng/mL, about 30 ng/ml to about 100 ng/ml, about 30 ng/ml to about 80 ng/mL, about 30 ng/ml to about 70 ng/ml, about 30 ng/ml to about 60 ng/mL, about 30 ng/ml to about 50 ng/mL or about 30 ng/ml to about 40 ng/mL.

Alternatively, the concentration of the growth factor having interleukin-like activity ranges from about 50 IU/mL to about 1,500 IU/mL, about 50 IU/mL to about 1,400 IU/mL, about 50 IU/mL to about 1,300 IU/mL, about 50 IU/mL to about 1,200 IU/mL, about 50 IU/mL to about 1,100 IU/mL, about 50 IU/mL to about 1,000 IU/mL, about 70 IU/mL to about 1,500 IU/mL, about 70 IU/mL to about 1,400 IU/mL, about 70 IU/mL to about 1,300 IU/mL, about 70 IU/mL to about 1,200 IU/mL, about 70 IU/mL to about 1,100 IU/mL, about 70 IU/mL to about 1,000 IU/mL, 80 IU/mL to about 1,500 IU/mL, about 80 IU/mL to about 1,400 IU/mL, about 80 IU/mL to about 1,300 IU/mL, about 80 IU/mL to about 1,200 IU/mL, about 80 IU/mL to about 1,100 IU/mL, about 80 IU/mL to about 1,000 IU/mL, about 90 IU/mL to about 1,500 IU/mL, about 90 IU/mL to about 1,400 IU/mL, about 90 IU/mL to about 1,300 IU/mL, about 90 IU/mL to about 1,200 IU/mL, about 90 IU/mL to about 1,100 IU/mL, about 90 IU/mL to about 1,000 IU/mL, about 100 IU/mL to about 1,500 IU/mL, about 100 IU/mL to about 1,400 IU/mL, about 100 IU/mL to about 1,300 IU/mL, about 100 IU/mL to about 1,200 IU/mL, about 100 IU/mL to about 1,100 IU/mL or about 100 IU/mL to about 1,000 IU/mL.

Preferably, the medium is free of serum. Examples of the medium include, but are not limited to, RPMI medium, TexMACS medium, IMDM medium, DMEM medium, CTS OpTMizer medium, KBM581 medium, AIM-V medium or X-VIVO medium, which can be used for culture by adding zoledronic acid and one or more growth factors having interleukin-like activity.

Optionally, Vδ²⁺ γδ T cells in the γδ T cell population having enriched Vδ²⁺ γδ T cell subpopulation can be further enriched by using immunomagnetic separation to remove the non-Vδ²⁺ γδ T cells in the population or subpopulation.

The γδ T cell population having an enriched Vδ²⁺ γδ T cell subpopulation is further expanded for 7-28 days and then the resulting cell population is harvested.

The cell population produced from the method of the present disclosure comprises an enriched Vδ²⁺ γδ T cell subpopulation. For example, the enriched γδ T cell population comprises at least about 85%, 88%, 90%, 91%, 92%, 93%, 94%, 95%, 96%, 97%, 98% or 99% Vδ²⁺ T cells with over about 70%, 75%, 80%, 85%, 90%, 91%, 92%, 93%, 94%, 95%, 96%, 97%, 98% or 99% post-expansion viability.

The Vδ²⁺ γδ T cell population of the present disclosure may provide a clinical grade cell line bank capable of proliferation and differentiation for use in a large number of patients. In certain embodiments, the Vδ²⁺ γδ T cells that have been collected and processed can be stored in a cell bank for future applications. These cells may be preserved using cryopreservation agents like DMSO or CryoStor™ and maintained at controlled freezing rates in liquid nitrogen. The γδ T cells can be organized into unitized storage, with specific units or dosages prepared for single or multiple treatment sessions.

The present disclosure also encompass Vδ²⁺ γδ T cells derived from the methods described herein for the purpose of modulating an immune response, treating infections, or addressing cancer as previously detailed.

For example, a method for treating a subject's infection or cancer is described, which involves administering the Vδ²⁺ γδ T cell population of the present disclosure. In this approach, the Vδ²⁺ γδ T cell population of the present disclosure is utilized to address various conditions, including viral, bacterial, fungal, or protozoan infections, as well as cancers in a subject.

In embodiments, the virus can be hepatitis B, hepatitis C, influenza (for example, a pandemic influenza virus from birds or pigs, such as H5N1, H7N3, H7N7, H7N9 and H9N2 (bird subtype) or H1N1, H1N2, H2N1, H3N1, H3N2H2N3 (pig subtype)), herpes variant, cytomegalovirus (CMV), Epsteiner-Bar virus, varicella, papillomavirus, Ebola, varicella-zoster virus, or natural pox.

In embodiments, the cancer can be sarcoma, basal cell skin cancer, lymphoma, leukemia, lymphoproliferative disorder, plasmacytoma, histiocytoma, adenoma, carcinomas of solid tissues, hypoxic tumor, genitourinary cancer, hematopoietic cancer, nervous system cancer, bile duct cancer, cervical cancer, squamous cell cancer, endometrial cancer, esophageal cancer, head and neck cancer, kidney cancer, liver cancer, lung cancer, melanoma, Merkel cell cancer, mesothelioma, stomach cancer, breast cancer or triple-negative breast cancer.

In certain embodiments, subjects receiving the Vδ²⁺ γδ T cell population of the present disclosure may also be given immunosuppressive agents either simultaneously, continuously, or at separate times. The use of immunosuppressive agents may assist in mitigating any negative systemic reactions to the gamma delta T cells.

The present disclosure presents a method for vaccinating an animal by administering an effective dose of the Vδ²⁺ γδ T cell population of the present disclosure. These cells can be obtained through the method described in the present disclosure. This vaccine is suitable for use in immunocompromised patients or subjects at an increased risk of developing infectious diseases or cancer.

### EMBODIMENTS

Embodiment 1: A method for producing a cell population comprising enriched Vδ²⁺ γδ T cells, comprising:
providing peripheral blood mononuclear cells (PBMCs); optionally the Vδ²⁺ γδ T cells contained in the PBMCs are separated by immunomagnetic separation to obtain a Vδ²⁺ γδ T cell subpopulation;
culturing the PBMCs or Vδ²⁺ γδ T cell subpopulation in a medium containing zoledronic acid and one or more growth factors having interleukin-like activity to specifically enrich Vδ²⁺ γδ T cells to obtain an enriched Vδ²⁺ γδ T cell population; and
harvesting the expanded Vδ²⁺ γδ T cell population.

Embodiment 2: A method for producing a cell population comprising enriched Vδ²⁺ γδ T cells, comprising:
(a1) providing peripheral blood mononuclear cells (PBMCs);
(b1) culturing the PBMCs in a medium containing zoledronic acid and one or more growth factors having interleukin-like activity to specifically enrich Vδ²⁺ γδ T cells to obtain an enriched Vδ²⁺ γδ T cell population; and
(c1) harvesting the expanded Vδ²⁺ γδ T cell population; or
(a2) providing peripheral blood mononuclear cells (PBMCs);
(ii) separating Vδ2⁺ γδ T cells by immunomagnetic separation to obtain a Vδ2⁺ γδ T cell subpopulation;
(b2) culturing the PBMCs in a medium containing zoledronic acid and one or more growth factors having interleukin-like activity to specifically enrich Vδ²⁺ γδ T cells to obtain an enriched Vδ²⁺ γδ T cell population; and
(c2) harvesting the expanded Vδ²⁺ γδ T cell population

Embodiment 3: The method of any one of preceding embodiments, wherein after step (b), (b1) or (b2), the method further comprises a step (e) of separating the enriched Vδ²⁺ γδ T cell population by immunomagnetic separation to remove non-Vδ²⁺ γδ T cells.

Embodiment 4: The method of any one of preceding embodiments, wherein after step (b), (b1), (b2) or (e), the method further comprises a step (f) of expanding the enriched Vδ²⁺ γδ T cell population by culturing it for 7-28 days to obtain the expanded Vδ²⁺ γδ T cell population.

Embodiment 5: The embodiments of any one of preceding embodiments, wherein the Vδ²⁺ γδ cells in the Vδ²⁺ γδ T cell population are unmodified.

Embodiment 6: The method of any one of preceding embodiments, wherein the PBMCs are obtained from a peripheral blood or umbilical cord blood or fractions thereof.

Embodiment 7: The method of any one of preceding embodiments, wherein the zoledronic acid is at a concentration of about 1 to about 30 μM, about 1 to about 28 μM, about 1 to about 25 μM, about 1 to about 23 μM, about 1 to about 20 μM, about 1 to about 18 μM, about 1 to about 15 μM, about 1 to about 13 μM, about 1 to about 10 μM, about 1 to about 8 μM, about 1 to about 5 μM, about 3 to about 30 μM, about 3 to about 28 μM, about 3 to about 25 μM, about 3 to about 23 μM, about 3 to about 20 μM, about 3 to about 18 μM, about 3 to about 15 μM, about 3 to about 13 μM, about 3 to about 10 μM, about 3 to about 8 μM, about 3 to about 5 μM, about 5 to about 30 μM, about 5 to about 28 μM, about 5 to about 25 μM, about 5 to about 23 μM, about 5 to about 20 μM, about 5 to about 18 μM, about 5 to about 15 μM, about 5 to about 13 μM, about 5 to about 10 μM, about 8 to about 30 μM, about 8 to about 28 μM, about 8 to about 25 μM, about 8 to about 23 μM, about 8 to about 20 μM, about 8 to about 15 μM, about 8 to about 12 μM, about 8 to about 10 μM, about 10 to about 30 μM, about 10 to about 28 μM, about 10 to about 25 μM, about 10 to about 23 μM, about 10 to about 20 μM, about 10 to about 18 μM, or about 10 to about 15 μM.

Embodiment 7: The method of any one of preceding embodiments, wherein the medium is free of serum.

Embodiment 8: The method of any one of preceding embodiments, wherein the growth factor having interleukin-like activity is interleukin.

Embodiment 9: The method of any one of preceding embodiments, wherein the concentration of the growth factor having interleukin-like activity ranges from about 5 ng/ml to about 200 ng/ml or about 50 IU/mL to about 1,500 IU/mL.

Embodiment 10: The method of any one of preceding embodiments, wherein the concentration of the growth factor having interleukin-like activity ranges from about 5 ng/ml to about 200 ng/mL, about 5 ng/ml to about 180 ng/ml, about 5 ng/ml to about 150 ng/mL, about 5 ng/ml to about 100 ng/mL, about 5 ng/ml to about 80 ng/mL, about 5 ng/ml to about 70 ng/mL, about 5 ng/mL to about 60 ng/ml, about 5 ng/ml to about 50 ng/ml, about 5 ng/ml to about 40 ng/ml, about 5 ng/ml to about 30 ng/mL, about 5 ng/ml to about 20 ng/ml, about 10 ng/ml to about 200 ng/ml, about 10 ng/ml to about 180 ng/mL, about 10 ng/ml to about 150 ng/mL, about 10 ng/ml to about 100 ng/ml, about 10 ng/mL to about 80 ng/mL, about 10 ng/ml to about 70 ng/ml, about 10 ng/ml to about 60 ng/ml, about 10 ng/ml to about 50 ng/mL, about 10 ng/mL to about 40 ng/mL, about 10 ng/ml to about 30 ng/mL, about 20 ng/ml to about 200 ng/ml, about 20 ng/mL to about 180 ng/mL, about 20 ng/ml to about 150 ng/mL, about 20 ng/mL to about 100 ng/ml, about 20 ng/ml to about 80 ng/mL, about 20 ng/ml to about 70 ng/ml, about 20 ng/mL to about 60 ng/ml, about 20 ng/mL to about 50 ng/ml, about 20 ng/mL to about 40 ng/ml, about 20 ng/ml to about 30 ng/mL, about 30 ng/ml to about 200 ng/mL, about 30 ng/ml to about 180 ng/ml, about 30 ng/ml to about 150 ng/ml, about 30 ng/ml to about 100 ng/ml, about 30 ng/mL to about 80 ng/ml, about 30 ng/mL to about 70 ng/ml, about 30 ng/ml to about 60 ng/ml, about 30 ng/ml to about 50 ng/ml or about 30 ng/ml to about 40 ng/ml.

Embodiment 11: The method of any one of preceding embodiments, wherein the concentration of the growth factor having interleukin-like activity ranges from about 50 IU/mL to about 1,500 IU/mL, about 50 IU/mL to about 1,400 IU/mL, about 50 IU/mL to about 1,300 IU/mL, about 50 IU/mL to about 1,200 IU/mL, about 50 IU/mL to about 1,100 IU/mL, about 50 IU/mL to about 1,000 IU/mL, about 70 IU/mL to about 1,500 IU/mL, about 70 IU/mL to about 1,400 IU/mL, about 70 IU/mL to about 1,300 IU/mL, about 70 IU/mL to about 1,200 IU/mL, about 70 IU/mL to about 1,100 IU/mL, about 70 IU/mL to about 1,000 IU/mL, 80 IU/mL to about 1,500 IU/mL, about 80 IU/mL to about 1,400 IU/mL, about 80 IU/mL to about 1,300 IU/mL, about 80 IU/mL to about 1,200 IU/mL, about 80 IU/mL to about 1,100 IU/mL, about 80 IU/mL to about 1,000 IU/mL, about 90 IU/mL to about 1,500 IU/mL, about 90 IU/mL to about 1,400 IU/mL, about 90 IU/mL to about 1,300 IU/mL, about 90 IU/mL to about 1,200 IU/mL, about 90 IU/mL to about 1,100 IU/mL, about 90 IU/mL to about 1,000 IU/mL, about 100 IU/mL to about 1,500 IU/mL, about 100 IU/mL to about 1,400 IU/mL, about 100 IU/mL to about 1,300 IU/mL, about 100 IU/mL to about 1,200 IU/mL, about 100 IU/mL to about 1,100 IU/mL or about 100 IU/mL to about 1,000 IU/mL.

Embodiment 12: The method of any one of preceding embodiments, wherein the interleukin is IL-2, IL-4, IL-7, IL-15, IL-21, interleukin-1β or any combination thereof.

Embodiment 13: The method of any one of preceding embodiments, wherein the interleukin is IL-2, IL-15, or a combination of IL-2 and IL-5.

Embodiment 14: The method of any one of preceding embodiments, wherein the interleukin is IL-2 in a concentration of about 100 IU/mL to about 1,000 IU/mL.

Embodiment 15: The method of any one of preceding embodiments, wherein the interleukin is IL-15 in a concentration of about 10 ng/ml to about 100 ng/mL.

Embodiment 16: The method of any one of preceding embodiments, wherein the interleukin is about 100 IU/mL to about 1,000 IU/mL of IL-2 in combination with about 10 ng/ml to about 100 ng/ml of IL-15.

Embodiment 17: The method of any one of preceding embodiments, wherein the medium containing IL-15 maintains a constant expansion of Vδ²⁺ γδ T-cells at a high level over time.

Embodiment 18: The method of any one of preceding embodiments, wherein the zoledronic acid mediated Vδ²⁺ γδ T-cell expansion can be increased by IL-15.

Embodiment 19: The method of any one of preceding embodiments, which comprises the steps of:
providing peripheral blood mononuclear cells (PBMCs); optionally the Vδ²⁺ γδ T cells contained in the PBMCs are separated by immunomagnetic separation to obtain a Vδ²⁺ γδ T cell subpopulation;
culturing the PBMCs or Vδ²⁺ γδ T cell subpopulation in a medium containing zoledronic acid at a concentration of about 5 μM to about 10 μM, about 5 μM or about 10 μM, and IL-12 at a concentration of about 100 IU/mL to about 1000 IU/mL, about 100 IU/mL or about 1000 IU/mL to specifically enrich Vδ²⁺ γδ T cells to obtain an enriched Vδ²⁺ γδ T cell population; and
harvesting the expanded Vδ²⁺ γδ T cell population.

Embodiment 20: The method of any one of preceding embodiments, which comprises the steps of:
providing peripheral blood mononuclear cells (PBMCs); optionally the Vδ²⁺ γδ T cells contained in the PBMCs are separated by immunomagnetic separation to obtain a Vδ²⁺ γδ T cell subpopulation;
culturing the PBMCs or Vδ²⁺ γδ T cell subpopulation in a medium containing zoledronic acid at a concentration of about 5 μM to about 10 μM, about 5 μM or about 10 μM, and IL-12 at a concentration of about 100 IU/mL to about 1000 IU/mL, about 100 IU/mL or about 1000 IU/mL to specifically enrich Vδ²⁺ γδ T cells to obtain an enriched Vδ²⁺ γδ T cell population;
optionally separating the enriched Vδ²⁺ γδ T cell population by immunomagnetic separation to remove non-Vδ²⁺ γδ T cells;
expanding the enriched Vδ²⁺ γδ T cell population by culturing it for 7-28 days to obtain the expanded Vδ²⁺ γδ T cell population; and
harvesting the expanded Vδ²⁺ γδ T cell population.

Embodiment 21: The method of any one of preceding embodiments, which comprises the steps of:
providing peripheral blood mononuclear cells (PBMCs); optionally the Vδ²⁺ γδ T cells contained in the PBMCs are separated by immunomagnetic separation to obtain a Vδ²⁺ γδ T cell subpopulation;
culturing the PBMCs or Vδ²⁺ γδ T cell subpopulation in a medium containing zoledronic acid at a concentration of about 5 μM to about 10 μM, about 5 μM or about 10 μM, and about 100 IU/mL to about 1000 IU/mL, about 100 IU/mL or about 1000 IU/mL of IL-12 in combination with about 100 IU/mL to about 1000 IU/mL, about 100 IU/mL or about 1000 IU/mL of IL-15 to specifically enrich Vδ²⁺ γδ T cells to obtain an enriched Vδ²⁺ γδ T cell population; and
harvesting the expanded Vδ²⁺ γδ T cell population.

Embodiment 22: The method of any one of preceding embodiments, which comprises the steps of:
providing peripheral blood mononuclear cells (PBMCs); optionally the Vδ²⁺ γδ T cells contained in the PBMCs are separated by immunomagnetic separation to obtain a Vδ²⁺ γδ T cell subpopulation;
culturing the PBMCs or Vδ²⁺ γδ T cell subpopulation in a medium containing zoledronic acid at a concentration of about 5 μM to about 10 μM, about 5 μM or about 10 μM, and about 100 IU/mL to about 1000 IU/mL, about 100 IU/mL or about 1000 IU/mL of IL-12 in combination with about 100 IU/mL to about 1000 IU/mL, about 100 IU/mL or about 1000 IU/mL of IL-15 to specifically enrich Vδ²⁺ γδ T cells to obtain an enriched Vδ²⁺ γδ T cell population;
optionally separating the enriched Vδ²⁺ γδ T cell population by immunomagnetic separation to remove non-Vδ²⁺ γδ T cells;
expanding the enriched Vδ²⁺ γδ T cell population by culturing it for 7-28 days to obtain the expanded Vδ²⁺ γδ T cell population; and
harvesting the expanded Vδ²⁺ γδ T cell population.

Embodiment 23: The method of any one of preceding embodiments, wherein the medium further comprises synthetic growth factors and amino acids tailored for T cell expansion; preferably the growth factor is neurotrophic factor (BDNF), epidermal growth factor (EGF), fibroblast growth factor (FGF), insulin-like growth factor (IGF-1), erythropoietin (EPO), vascular growth factor (VEGF), transforming growth factor beta (TGF-β), nerve growth factor (NGF) or platelet derived growth factors (PDGF); preferably the amino acid is glutamine (Gln), alanine (Ala), serine (Ser), leucine (Leu), methionine (Met), arginine (Arg), cysteine (Cys) or cystine (Cys-Cys).

Embodiment 24: The method of any one of preceding embodiments, wherein the step of expanding the Vδ²⁺ γδ T cells is conducted under 37° C. and 5% CO₂.

Embodiment 25: The method of any one of preceding embodiments, wherein the harvested Vδ²⁺ γδ T cells are cryopreserved in a solution containing DMSO and a protective cryoprotectant.

Embodiment 26: A cell population produced from a method of any one of preceding embodiments.

Embodiment 27: A Vδ²⁺ γδ T cell population, comprising enriched Vδ²⁺ γδ T cells.

Embodiment 28: The Vδ²⁺ γδ T cell population of Embodiment 27, wherein the Vδ²⁺ γδ T cell population comprises at least about 85%, 88%, 90%, 91%, 92%, 93%, 94%, 95%, 96%, 97%, 98% or 99% Vδ²⁺ γδ T cells with over about 70%, 75%, 80%, 85%, 90%, 91%, 92%, 93%, 94%, 95%, 96%, 97%, 98% or 99% post-expansion viability.

Embodiment 29: The Vδ²⁺ γδ T cell population of Embodiment 26, 27 or 28, the Vδ²⁺ γδ T cell population described herein is in therapeutic-grade.

Embodiment 30: The Vδ²⁺ γδ T cell population of any one of Embodiments 26 to 29, wherein the Vδ²⁺ γδ T cell population has low residual αβ T cells.

Embodiment 31: The Vδ²⁺ γδ T cell population of any one of Embodiments 26 to 30, wherein the enriched γδ T cell population has residual αβ T cell less than about 0.5%, 1%, 2%, 3%, 4%, 5%, 6%, 7%, 8%, 9% or 10%.

Embodiment 32: The Vδ²⁺ γδ T cell population of any one of Embodiments 26 to 31, wherein the Vδ²⁺ γδ T cell population comprises at least about 95%, 96%, 97%, 98% or 99% Vδ²⁺ γδ T cells with over about 90%, 91%, 92%, 93%, 94%, 95%, 96%, 97%, 98% or 99% post-expansion viability.

Embodiment 33: The Vδ²⁺ γδ T cell population of any one of Embodiments 26 to 32, wherein the Vδ²⁺ γδ T cells express one or more CD3, CD45 and NKG2D in an intensity higher than about 70%, 75%, 80%, 85%, 90%, 95%, 96%, 97%, 98%, 98.5%, 99% or 99.5%.

Embodiment 34: The Vδ²⁺ γδ T cell population of any one of Embodiments 26 to 33, wherein the Vδ²⁺ γδ T cells express NKG2D in an intensity higher than about 95%, 96%, 97%, 98%, 98.5%, 99% or 99.5%. In a further embodiment, the Vδ²⁺ γδ T cells described herein express NKG2D in an intensity higher than about 98%.

Embodiment 35: The Vδ²⁺ γδ T cell population of any one of Embodiments 26 to 34, wherein the Vδ²⁺ γδ T cells express CD3 in an intensity higher than about 95%, 96%, 97%, 98%, 98.5%, 99% or 99.5%. In a further embodiment, the Vδ²⁺ γδ T cells described herein express CD3 in an intensity higher than about 99%.

Embodiment 36: The Vδ²⁺ γδ T cell population of any one of Embodiments 26 to 35, wherein the Vδ²⁺ γδ T cells express CD45 in an intensity higher than about 95%, 96%, 97%, 98%, 98.5%, 99% or 99.5%. In a further embodiment, the Vδ²⁺ γδ T cells described herein express CD45 in an intensity higher than about 98%.

Embodiment 37: The Vδ²⁺ γδ T cell population of any one of Embodiments 26 to 36, wherein the Vδ²⁺ γδ T cells express CD3, CD45 and NKG2D in an intensity higher than about 70%, 75%, 80%, 85%, 90%, 95%, 96%, 97%, 98%, 98.5%, 99% or 99.5%. In a further embodiment, the Vδ²⁺ γδ T cells described herein express CD3, CD45 and NKG2D in an intensity higher than about 95%, 96%, 97%, 98%, 98.5%, 99% or 99.5%.

Embodiment 38: A method of modulating an immune response in a subject, comprising administering the enriched Vδ²⁺ γδ T cell population of any one of Embodiments 26 to 37 to the subject.

Embodiment 39: A method for use in immunotherapy in a subject, comprising administering the enriched Vδ²⁺ γδ T cell population of any one of Embodiments 26 to 37 to the subject.

Embodiment 40: The method of Embodiment 39, wherein the immunotherapy provides multifunctional immune modulation.

Embodiment 41: The method of Embodiment 39 or 40, wherein the immunotherapy provides potent effector functionality and cytokine-producing capability of the expanded Vδ²⁺ γδ T cells upon tumor cell recognition.

Embodiment 42: The method of Embodiment 39, 40 or 41, wherein the immunotherapy is chimeric antigen receptor (CAR) T cell therapy.

Embodiment 43: A method for treating a cancer in a subject, comprising administering the enriched Vδ²⁺ γδ T cell population described herein to the subject.

Embodiment 44: The method of Embodiment 43, wherein the cancer is sarcoma, basal cell skin cancer, lymphoma, leukemia, lymphoproliferative disorder, plasmacytoma, histiocytoma, adenoma, carcinomas of solid tissues, hypoxic tumor, genitourinary cancer, hematopoietic cancer, nervous system cancer, bile duct cancer, cervical cancer, squamous cell cancer, endometrial cancer, esophageal cancer, head and neck cancer, glioblastoma (GBM) (such as glioblastoma multiforme), kidney cancer, hepatocellular carcinoma, liver cancer, lung cancer, pancreatic cancer, melanoma, Merkel cell cancer, mesothelioma, stomach cancer, breast cancer, triple-negative breast cancer.

Embodiment 45: The method of Embodiment 43 or 44, wherein the cancer is breast cancer, lung cancer, hepatocellular carcinoma, liver cancer, pancreatic cancer or GBM.

Embodiment 46: The method of Embodiment 43 or 44, wherein the leukemia is chronic lymphocytic leukemia, chronic myelogenous leukemia, acute myelogenous leukemia, acute lymphoblastic leukemia, or T cell and B cell leukemias; the lymphoma is Hodgkin's lymphoma or non-Hodgkin lymphoma; and the genitourinary cancer is cervical cancer or bladder cancer.

Embodiment 47: A method for treating an infectious disease in a subject, comprising administering the enriched Vδ²⁺ γδ T cell population described herein to the subject. In some embodiment, the infectious disease is bacterial infections such as those caused by mycobacteria (e.g. tuberculosis), viral infections such as those caused by herpes simplex viruses (HSV), human immunodeficiency viruses (HIV), the hepatitis viruses, SARS-CoV-2 viruses, papillomaviruses (HPV), Epstein-Barr viruses (EBV), measles viruses, cytomegaloviruses (CMV), hepatitis C viruses (HCV), or influenza viruses, and parasitic infections such as those caused by plasmodium (e.g. malaria).

Embodiment 48: The method of Embodiment 47, wherein the infectious disease is a viral infection.

Embodiment 49: The method of Embodiment 48, wherein the viral infection is caused by human immunodeficiency virus (HIV), SARS-CoV-2, papillomavirus (HPV), herpes simplex virus (HSV), Epstein-Barr virus (EBV), measles virus, cytomegalovirus (CMV), hepatitis C virus (HCV), or influenza virus.

Embodiment 50: A method for vaccinating an animal comprising administering the enriched Vδ²⁺ γδ T cell population described herein to the animal.

The present invention is described in greater detail in the examples presented below, which are preceded by a brief description of the figures. It goes without saying however, that these examples are given by way of illustration of the subject of the invention and do not constitute in any manner a limitation thereto.

### EXAMPLES

### Example 1 Zoledronic Acid Mediates γδ T-Cell Expansion at Different IL-2 Concentrations Depending on the Concentrations of Zoledronic Acid

### Materials and Methods

PBMC isolation: Peripheral blood mononuclear cells (PBMCs) were isolated from fresh human peripheral blood or leukopak collected from healthy donors using Ficoll-Paque (Cytiva) density-gradient centrifugation. After separation, PBMCs were washed twice with PBS and resuspended in KBM581 (Kohjin Bio) medium containing 10% human platelet lysate (HPL; EliteGro).

Initial cell seeding: PBMCs were seeded at 1×10⁶ cells/mL in culture flasks.

Zoledronic acid stimulation: Zoledronic acid (ZOL; Novartis) stock solution was diluted in the culture medium to final concentrations of 5 μM and 10 μM.

IL-2 supplementation: Recombinant human IL-2 (Akron Bio) was added to the culture at the following concentrations: 100 IU/mL, 1000 IU/mL.

Culture conditions: Cells were incubated at 37° C., 5% CO₂ for 14 days. Cell density was maintained between 0.5-2×10⁶ cells/mL by adding fresh medium every 2-3 days. ZOL and IL-2 were added at Day 0. Fresh IL-2 was replenished every 2-3 days during culture.

Flow cytometric analysis: On Day 14, cells were harvested and stained with: anti-CD3 (Sony), anti-Vδ2 (Sony), Zombie B550 viability dye (Sony). Cells were analyzed using a Sony SA3800 Spectral Analyzer. Vδ²⁺ γδ T-cell expansion was quantified as: Fold expansion=Total γδ T cells on Day 14/Total γδ T cells on Day 0.

It is shown that Vδ²⁺ γδ T-cell expansion in the presence of zoledronic acid was achieved at different IL-2 concentrations. Furthermore, lower concentration of zoledronic acid to Vδ²⁺ γδ T-cells resulted in greater Vδ²⁺ γδ T-cell expansion, indicating that the induction of Vδ²⁺ γδ T-cell expansion by zoledronic acid is dose dependent (FIG. 1).

### Example 2 Effect of Different Cytokine Combinations on Zoledronic Acid Mediated γδ T-Cell Expansion

### Materials and Methods

PBMC isolation: PBMCs were isolated from fresh human peripheral blood or leukopak collected from healthy donors using Ficoll-Paque density-gradient centrifugation. After separation, PBMCs were washed twice with PBS and resuspended in KBM581 medium containing 10% HPL.

Cell seeding: PBMCs were seeded at 1×10⁶ cells/mL in culture flasks.

ZOL stimulation: ZOL stock solution was diluted in culture medium to reach a final concentration of 5 μM, added on Day 0.

Cytokine supplementation conditions: Three cytokine conditions were tested: (1) IL-2 alone: IL-2 (1000 IU/mL), (2) IL-15 (Akron Bio) alone: IL-15 (10 ng/ml), (3) IL-2+IL-15 combination: IL-2 (1000 IU/mL)+IL-15 (10 ng/ml).

Culture conditions: Cells were incubated at 37° C., 5% CO₂ for 14 days. Cell density was maintained between 0.5-2×10⁶ cells/mL by adding fresh medium every 2-3 days. ZOL and cytokines were added at Day 0. Fresh cytokines were replenished every 2-3 days during culture.

Flow cytometric analysis: On Day 14, cells were harvested and stained with: anti-CD3, anti-Vδ2, Zombie B550 viability dye. Cells were analyzed using a Sony SA3800 Spectral Analyzer. Vδ²⁺ γδ T-cell expansion was quantified as: Fold expansion=Total γδ T cells on Day 14/Total γδ T cells on Day 0.

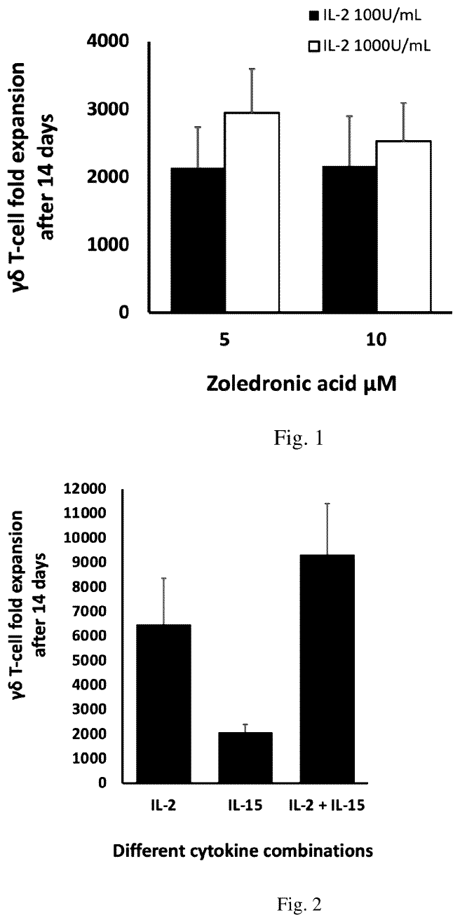

It is shown that combining IL-2 with IL-15 can significantly enhance the zoledronic acid mediated Vδ²⁺ γδ T-cell expansion (FIG. 2).

### Example 3: Zoledronic Acid Mediated γδ T-Cell Expansion at Different Concentrations of IL-15

### Materials and Methods

PBMC isolation: PBMCs were isolated from fresh human peripheral blood or leukopak collected from healthy donors using Ficoll-Paque density-gradient centrifugation. After separation, PBMCs were washed twice with PBS and resuspended in KBM581 medium containing 10% HPL.

Cell seeding: PBMCs were seeded at 1×10⁶ cells/mL in culture flasks.

ZOL stimulation: ZOL stock solution was diluted in culture medium to reach a final concentration of 5 μM, added on Day 0.

IL-15 supplementation: Recombinant human IL-15 was added at three different concentrations: 10 ng/ml, 30 ng/ml, 100 ng/ml.

Culture conditions: Cells were maintained at 37° C., 5% CO₂. Total culture duration was 14 days. ZOL and IL-15 were added at Day 0. Fresh IL-15 was replenished every 2-3 days. Fresh KBM581 medium was added every 2-3 days to maintain a density of 0.5-2×10⁶ cells/mL.

Flow cytometric analysis: On Day 14, cells were harvested and stained with: anti-CD3, anti-Vδ2, Zombie B550 viability dye. Cells were analyzed using a Sony SA3800 Spectral Analyzer. Vδ²⁺ γδ T-cell expansion was quantified as: Fold expansion=Total Vδ²⁺ γδ T cells on Day 14/Total Vδ²⁺ γδ T cells on Day 0.

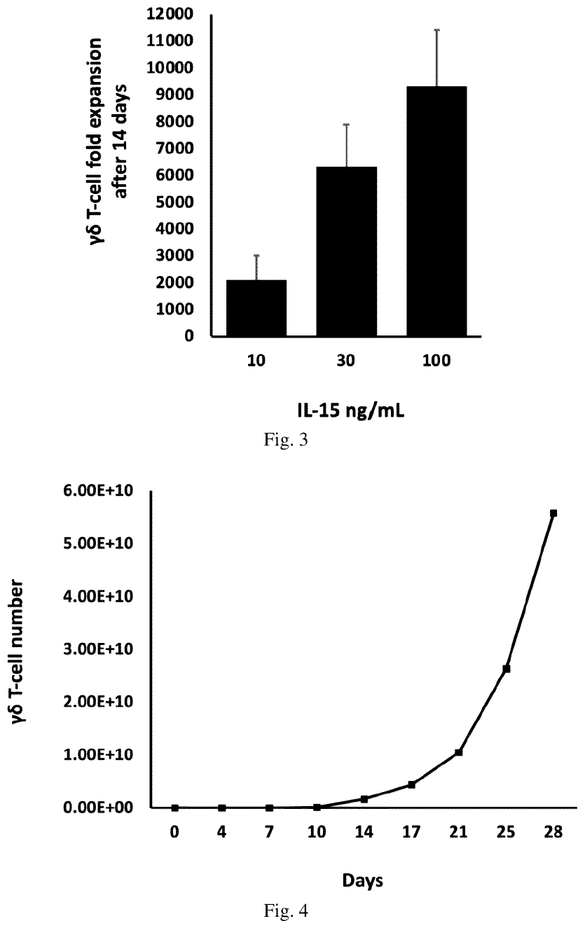

Zoledronic acid mediated Vδ²⁺ γδ T-cell expansion can be increased by IL-15 in a dose dependent manner (FIG. 3).

### Example 4 Constant Long-Term γδ T-Cell Expansion by Adding IL-15

### Materials and Methods

PBMC isolation: PBMCs were isolated from fresh human peripheral blood or leukopak collected from healthy donors using Ficoll-Paque density-gradient centrifugation. After separation, PBMCs were washed twice with PBS and resuspended in KBM581 medium containing 10% HPL.

Cell seeding: PBMCs were seeded at 1×10⁶ cells/mL in culture flasks.

ZOL stimulation: ZOL stock solution was diluted into culture medium to reach a final concentration of 5 μM, added on Day 0.

Cytokine supplementation for long-term expansion: IL-2 (1000 IU/mL), IL-15 (10 ng/mL).

Culture conditions: Cells were incubated at 37° C., 5% CO₂ for 28 days. Cell density was maintained between 0.5-2×10⁶ cells/mL by adding fresh medium every 2-3 days. ZOL and cytokines were added at Day 0. Fresh cytokines were replenished every 2-3 days during culture.

Cell counting and calculation of Vδ²⁺ γδ T cell numbers: Total cell numbers were quantified on Days 0, 4, 7, 10, 14, 17, 21, 25, and 28 using NucleoCounter NC-200 automated cell counter. In parallel, an aliquot of cells from each timepoint was analyzed by flow cytometry to determine the proportion of Vδ²⁺ γδ T cells within the culture. Cells were stained with anti-CD3, anti-Vδ2, Zombie B550 viability dye, and the percentage of viable Vδ²⁺ γδ T cells was recorded. Cells were analyzed using a Sony SA3800 Spectral Analyzer. The absolute number of Vδ²⁺ γδ T cells at each timepoint was calculated according to the following formula: Absolute Vδ²⁺ γδ T cell count=Total viable cells×Percentage of Vδ²⁺ γδ T cells.

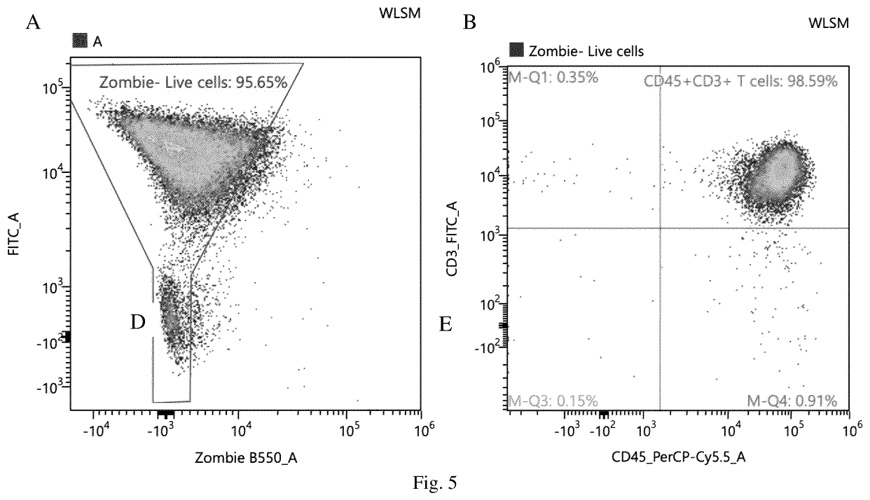

It was possible to maintain a constant expansion of Vδ²⁺ γδ T-cells at a high level over long period of time by adding IL-15 during the cultivation (FIG. 4).

### Example 5: Isolation and Expansion of GDT Cells

### Materials and Methods

PBMC isolation: PBMCs were isolated from fresh human peripheral blood or leukopak collected from healthy donors using Ficoll-Paque density-gradient centrifugation. After separation, PBMCs were washed twice with PBS and resuspended in KBM581 medium containing 10% HPL.

Cell seeding: PBMCs were seeded at 1×10⁶ cells/mL in culture flasks.

ZOL stimulation: ZOL stock solution was diluted in culture medium to reach a final concentration of 5 μM, added on Day 0.

IL-2 supplementation: Recombinant human IL-2 was added at 1000 IU/mL.

Culture conditions: Cells were incubated at 37° C., 5% CO₂ for 14 days. Cell density was maintained between 0.5-2×10⁶ cells/mL by adding fresh medium every 2-3 days. ZOL and IL-2 were added at Day 0. Fresh IL-2 was replenished every 2-3 days during culture.

αβ T cell Depletion: After 14 days of expansion, cells were harvested and subjected to TCRαβ depletion using the αβ T-cell Depletion Kit (Miltenyi Biotec) following the manufacturer's protocol.

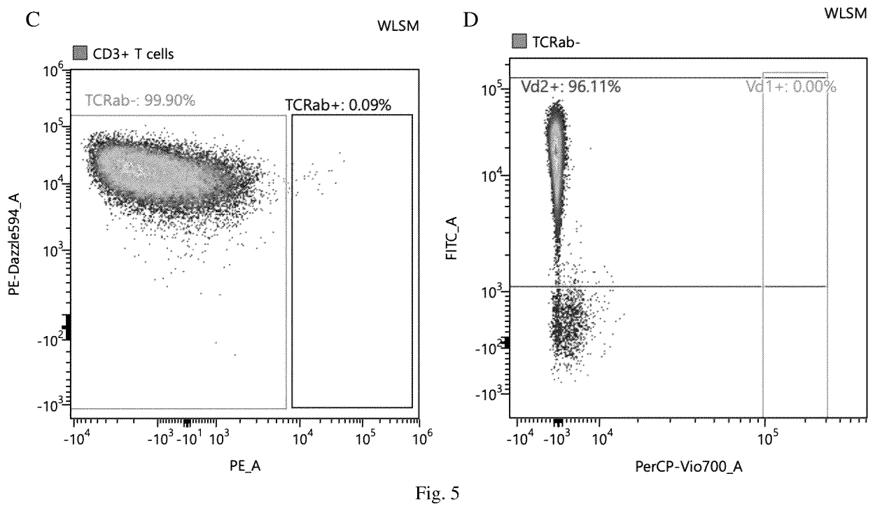

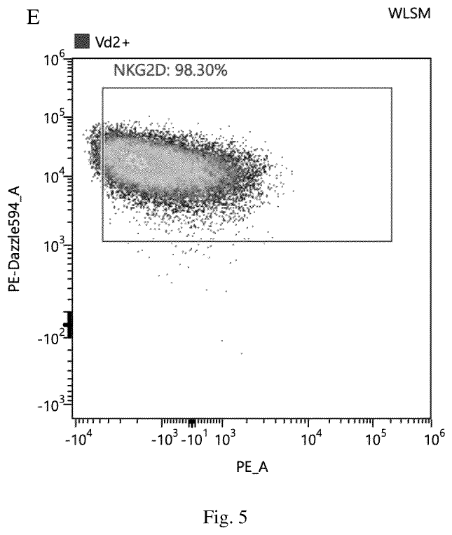

Flow Cytometry Analysis: Cells were stained with Zombie B550 viability dye (Sony), anti-CD3 (Sony), anti-CD45 (Sony), anti-TCRαβ (Sony), anti-Vδ2 (Sony), anti-Vδ1 (Miltenyi Biotec), anti-NKG2D (Sony). Cells were analyzed using a Sony SA3800 Spectral Analyzer. Gating strategy matched FIG. 5, including: Live cells (Zombie-), CD³⁺CD45⁺ T cells, TCRαβ population, Vδ1⁻Vδ2⁺ subsets, and NKG2D expression within Vδ2⁺ cells.

Peripheral blood mononuclear cells PBMCs) were isolated from healthy donors and enriched for Vδ2+ T cells using immunomagnetic separation. The cells were cultured in KBM581 supplemented with IL-2 1000 IU/mL) and zoledronic acid 5 μM). After 14 days of expansion, flow cytometry analysis revealed a 10,000-fold increase in cell numbers with a viability of >90%. The expanded cells exhibited high surface expression of NKG2D, indicating a potent cytotoxic phenotype. Additionally, the expanded cell population demonstrated low residual αβ T cell contamination, ensuring enhanced safety for allogeneic applications. These cells exhibited robust anti-tumor activity against various cancer cell lines, confirming their immunotherapeutic potential for clinical use (FIG. 5).

### Example 6 Functional Analysis of Expanded GDT Cells

### Materials and Methods

Tumor Cell Preparation: MDA-MB-231 (ATCC) breast cancer cells, HuH-7 (JCRB) liver cancer cells, and A549 (ATCC) lung cancer cells were expanded according to each cell line's recommended conditions. MDA-MB-231 cells were cultured in Leibovitz's L-15 medium supplemented with 10% fetal bovine serum (FBS) and maintained under CO₂-free incubation, whereas HuH-7 cells were maintained in low-glucose DMEM with 10% FBS and incubated at 37° C. with 5% CO₂. A549 cells were cultured in F-12K medium supplemented with 10% FBS under the same CO₂ conditions. All cell lines were confirmed to be free of mycoplasma contamination before use. For cytotoxicity assays, each tumor cell line was seeded at a density of 3×10⁵ cells per well in 6-well plates and allowed to attach for approximately 16 hours before Vδ²⁺ γδ T cell addition.

Co-Culture Setup with Vδ²⁺ γδ T Cells: Expanded Vδ²⁺ γδ T cells were washed and resuspended in complete medium prior to being added to the tumor cells. Co-cultures were prepared at effector-to-target ratios of 0:1, 1:1, 2:1, 5:1, and 10:1, corresponding to the addition of 0, 3×10⁵, 6×10⁵, 1.5×10⁶, and 3×10⁶ Vδ²⁺ γδ T cells per well, respectively. Following Vδ²⁺ γδ T-cell addition, the plates were incubated for 24 hours at 37° C. under the appropriate incubation conditions for each tumor type.

Cell Harvesting and Staining Procedure: After the 24-hour co-culture period, both suspended and adherent cells were collected for apoptosis assessment. Suspended cells were harvested first, and adherent tumor cells were washed twice with PBS and dissociated using 0.25% trypsin at 37° C. for approximately three minutes. The detached cells were washed twice with PBS and combined with the suspended fraction. To distinguish tumor cells from Vδ²⁺ γδ T cells, the mixed cell suspension was incubated with an APC/Cy7-conjugated anti-CD45 antibody for 20 minutes on ice in the dark, followed by washing and resuspension in Annexin V binding buffer. Annexin V-FITC and propidium iodide were then added, and the cells were incubated for 15 minutes at room temperature before flow cytometry analysis. Samples were analyzed using a CytoFLEX S flow cytometer, and tumor cells were identified as CD45− negative events. A minimum of 10,000 tumor cell events were acquired for each condition.

Flow Cytometry Analysis of Apoptosis: Apoptotic states were defined based on Annexin V and propidium iodide staining patterns, with viable cells identified as Annexin V⁻/PI⁻, early apoptotic cells as Annexin V⁺/PI⁻, late apoptotic cells as Annexin V⁺/PI⁺, and dead cells as Annexin V⁻/PI⁺. The proportion of cells in each apoptosis category was quantified within the CD45⁻ tumor cell population. These analyses enabled direct comparison of Vδ²⁺ γδ T cell-mediated cytotoxicity across different E:T ratios and tumor cell types.

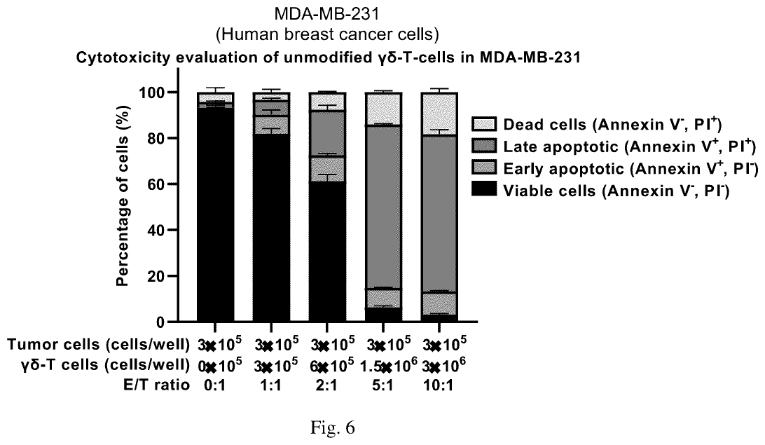

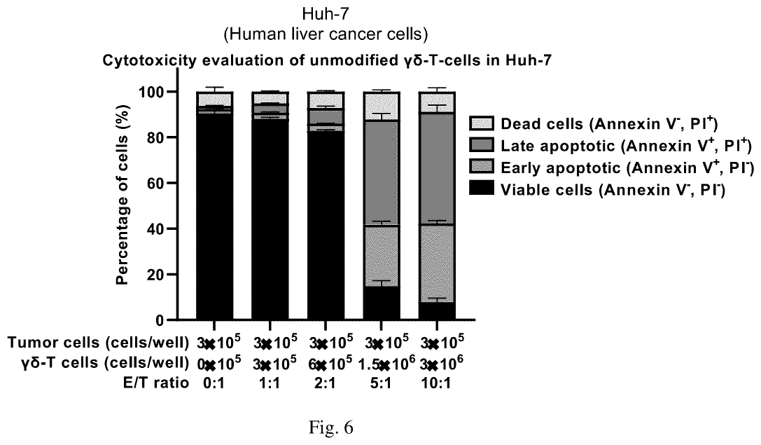

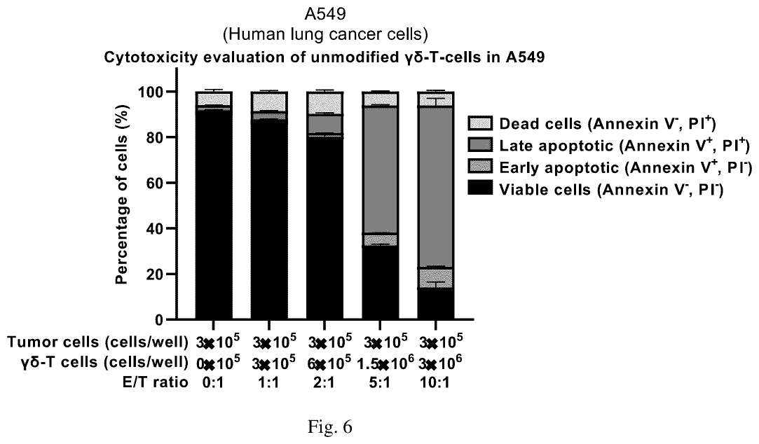

Expanded GDT cells were co-cultured with MDA-MB231 cells human breast cancer cells), Huh-7 cells human liver cancer cells), and A549 cells human lung cancer cells) at effector-to-target E/T) ratios of 0:1, 1:1, 2:1, 5:1, and 10:1. After 24 hours of co-culture, Annexin V/PI staining was performed to evaluate tumor cell apoptosis. The analysis demonstrated a dose-dependent cytotoxic effect across all three cancer cell lines. For MDA-MB231 and Huh-7 cells, significant tumor cell death was observed at an E/T ratio of 5:1, where over 80% of the cancer cells underwent apoptosis. This effect was further confirmed at a 10:1 ratio, showing consistent and robust cytotoxic activity. In contrast, A549 cells required a higher E/T ratio, with over 80% of the cells undergoing apoptosis only at a 10:1 ratio. These results highlight the potent and selective anti-tumor capabilities of the expanded GDT cells against multiple cancer types, supporting their potential in cancer immunotherapy applications (FIG. 6).

### Example 7 Cytokine Secretion Profile of Unmodified γδ T Cells Following Co-Culture with Tumor Target Cells

### Materials and Methods

Co-Culture of Vδ²⁺ γδ T Cells with Tumor Target Cells: Unmodified Vδ²⁺ γδ T cells were co-cultured with tumor target cells to evaluate cytokine secretion following tumor recognition. Tumor cells were seeded one day prior to effector addition to ensure stable adherence and were cultured under standard conditions until use. Expanded Vδ²⁺ γδ T cells were washed and resuspended in complete medium before being added to the tumor cells at effector-to-target (E:T) ratios of 0:1, 0.5:1, 1:1, and 2:1. The co-cultures were maintained for 24 hours at 37° C. and 5% CO₂ to allow interaction between effector and target cells and subsequent cytokine release into the culture supernatant.

Collection of Supernatants for Cytokine Quantification: Following the 24-hour co-culture period, cell culture supernatants were collected carefully to avoid disturbing the cell layer. The supernatants were transferred into sterile tubes and centrifuged briefly to remove cell debris. Clarified supernatants were then stored at −80° C. until cytokine analysis. This ensured preservation of cytokine integrity and prevented degradation prior to ELISA measurement.

Cytokine Detection by ELISA: The concentrations of IFN-γ, IL-4, IL-9, and TNF-α in the collected supernatants were quantified using commercially available human ELISA kits according to the manufacturer's instructions. Standard curves were generated for each cytokine, and sample concentrations were calculated based on optical density measurements obtained using a microplate reader. Each sample was measured in technical replicates to ensure data accuracy and reproducibility.

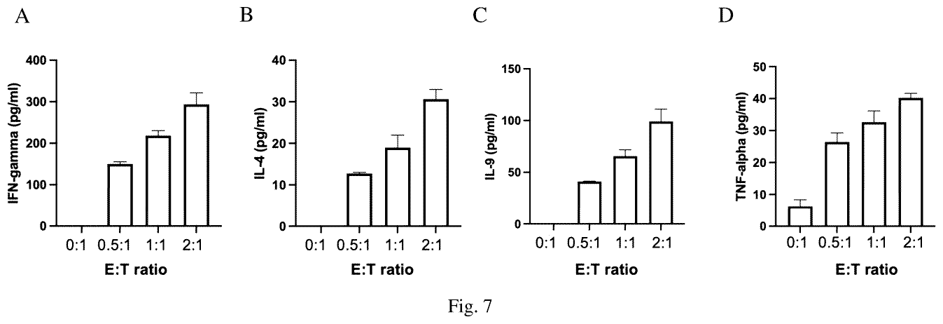

Unmodified γδ T cells were co-cultured with tumor cells at various effector-to-target (E:T) ratios (0:1, 0.5:1, 1:1, and 2:1) for 24 hours. The concentrations of IFN-γ, IL-4, IL-9, and TNF-α in the supernatants were quantified by ELISA. A dose-dependent increase in cytokine secretion was observed with increasing E:T ratios. Specifically, γδ T cells produced high levels of IFN-γ (>300 μg/mL) and TNF-α (>40 μg/mL), indicating robust Th1-type activation, as well as detectable IL-4 and IL-9 secretion, suggesting multifunctional immune modulation. These data confirm the potent effector functionality and cytokine-producing capability of the expanded γδ T cells upon tumor cell recognition (FIG. 7).

### Example 8 In Vivo Anti-Tumor Efficacy of Unmodified γδ T Cells in a Huh7 Hepatocellular Carcinoma Xenograft Model

Tumor Cell Preparation and Xenograft Establishment: Human hepatocellular carcinoma Huh-7 cells were expanded under standard culture conditions and harvested during logarithmic growth. Cells were washed with phosphate-buffered saline and resuspended in a 1:1 mixture of serum-free medium and Matrigel. Each mouse received a subcutaneous inoculation of 2×10⁶ Huh-7 cells in a 200-μL injection volume. Following implantation, tumor growth was monitored by caliper measurements, and tumor volume was calculated using the formula 0.5×length×width². Animals were observed daily to ensure stable tumor engraftment prior to treatment initiation.

Co-Inoculation Study for Dose-Dependent Tumor Inhibition: To evaluate the prophylactic anti-tumor effects of Vδ²⁺ γδ T cells, freshly expanded Vδ²⁺ γδ T cells were mixed directly with Huh-7 cells at the time of implantation. The mixtures were prepared at effector-to-target ratios of 0:1, 2:1, and 4:1, maintaining a constant tumor cell number of 2×10⁶ cells per injection. Tumor growth was measured throughout the study period. Mice receiving Huh-7 cells alone exhibited continuous and rapid tumor progression, whereas co-administration of Vδ²⁺ γδ T cells resulted in significant and ratio-dependent suppression of tumor formation. Complete inhibition of detectable tumor growth was observed in mice receiving Vδ²⁺ γδ T cells at the 4:1 ratio, demonstrating potent anti-tumor activity at the site of implantation.

Systemic Vδ²⁺ γδ T-Cell Administration in Established Tumors: A separate cohort was used to evaluate the therapeutic efficacy of Vδ²⁺ γδ T cells against established tumors. Treatment began when tumor sizes reached approximately 100 mm³, typically on Day 21 after implantation. Mice received intravenous injections of Vδ²⁺ γδ T cells via the tail vein at doses of either 5×10⁶ or 2×10⁷ cells per administration. Injections were performed once every three days in a recurring schedule. Tumor volumes were recorded throughout the treatment period. Animals treated systemically with Vδ²⁺ γδ T cells showed clear inhibition of tumor growth compared to PBS-treated controls, with the higher Vδ²⁺ γδ T-cell dose producing the most pronounced suppression. Representative excised tumors confirmed the reductions in tumor size observed in vivo.

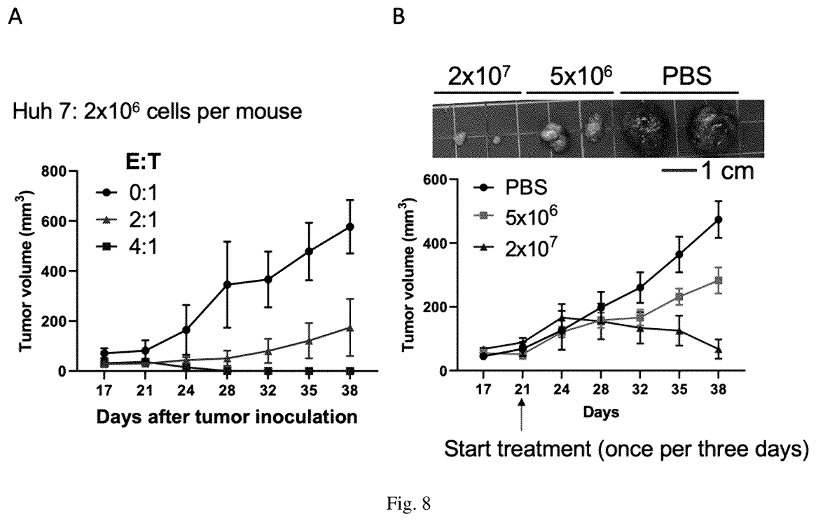

Each mouse received 2×10⁶ Huh7 cells subcutaneously, either alone (E:T=0:1) or co-injected with γδ T cells at E:T ratios of 2:1 and 4:1. Tumor volume was monitored over time. Mice receiving γδ T cells showed marked suppression of tumor growth in a dose-dependent manner, with complete inhibition observed at E:T=4:1. Treatment was initiated when tumors reached approximately 100 mm³ (Day 21). Mice were injected with 5×10⁶ or 2×10⁷ γδ T cells intravenously once every three days. Representative tumor images (top) and tumor growth curves (bottom) show significant tumor regression in the γδ T cell-treated groups compared to PBS controls. These data demonstrate that unmodified γδ T cells exert potent anti-tumor activity against hepatocellular carcinoma both in prophylactic and therapeutic settings (FIG. 8).

### Example 9 In Vivo Anti-Tumor Efficacy of Unmodified γδ T Cells in an A549 Lung Cancer Xenograft Model

Establishment of the A549 Xenograft Model: Human A549 lung adenocarcinoma cells were expanded under standard culture conditions and harvested during logarithmic growth. The cells were washed with phosphate-buffered saline, resuspended in serum-free medium, and mixed with an equal volume of Matrigel to support tumor establishment. Each immunodeficient mouse received a subcutaneous injection of 2×10⁶ A549 cells in a total volume of 200 μL. Tumor implantation sites were monitored three times weekly, and tumor size was measured using calipers. Tumor volume was calculated using the standard formula 0.5×length×width².

Co-Injection Study for Evaluation of Local Vδ²⁺ γδ T-Cell Activity: To evaluate the direct anti-tumor activity of unmodified Vδ²⁺ γδ T cells at the site of tumor initiation, Vδ²⁺ γδ T cells were freshly expanded and resuspended in complete medium prior to injection. A549 tumor cells were combined with Vδ²⁺ γδ T cells at an A549:T-cell ratio of 1:2, maintaining the tumor cell inoculum at 2×10⁶ cells per mouse. The mixtures were injected subcutaneously into the flank region, and tumor progression was monitored at regular intervals. Mice receiving A549 cells alone exhibited progressive tumor growth, whereas those co-injected with Vδ²⁺ γδ T cells demonstrated rapid and sustained inhibition of tumor formation. Tumor regression was observed as early as Day 17, and near-complete disappearance of measurable tumor masses occurred by Day 24. The difference in tumor volume between treated and untreated groups was statistically significant (***p<0.001), demonstrating potent Vδ²⁺ γδ T-cell-mediated suppression of A549 tumor growth.

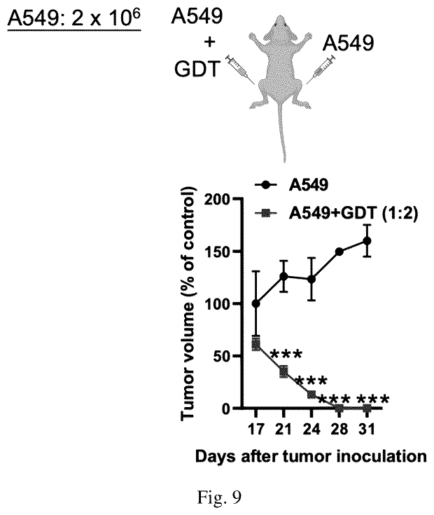

A549 cells (2×10⁶) were subcutaneously implanted in immunodeficient mice, either alone or co-injected with Vδ²⁺ γδ T cells at an A549:T ratio of 1:2. Tumor volume was measured every three to four days. Mice receiving γδ T cells showed a rapid and sustained suppression of tumor growth, with near-complete tumor regression by Day 24 compared with the A549-alone group (***p<0.001). These findings demonstrate that unmodified Vδ²⁺ γδ T cells exhibit potent in vivo cytotoxic activity against human lung cancer through local delivery routes. The unmodified Vδ²⁺ γδ T cells markedly inhibited tumor growth in the co-injection model (FIG. 9).

### Example 10: Cytotoxic Activity of Unmodified γδ T Cells Against BxPC-3 Pancreatic Cancer Cells Measured by Real-Time Cell Analysis

Preparation of BxPC-3 Pancreatic Cancer Cells: BxPC-3 human pancreatic cancer cells were cultured under standard conditions in complete growth medium until they reached logarithmic phase. Cells were harvested, washed with phosphate-buffered saline, and resuspended in fresh culture medium prior to seeding. For real-time cytotoxicity monitoring, cells were plated into E-Plate 16 plates compatible with the xCELLigence Real-Time Cell Analysis system. A total of 1×10⁴ BxPC-3 cells were added per well, and the plates were inserted into the xCELLigence station to allow continuous impedance-based monitoring. Cells were permitted to attach and proliferate until a stable baseline cell index was achieved.

Co-Culture of BxPC-3 Cells with Vδ²⁺ γδ T Cells: Unmodified Vδ²⁺ γδ T cells were expanded ex vivo and prepared for co-culture by washing and resuspending them in complete medium. Once BxPC-3 cells reached stable adhesion, Vδ²⁺ γδ T cells were added directly into the wells at tumor-to-effector ratios of 1:2, 1:3, and 1:4. Co-cultures were maintained at 37° C. with continuous impedance recording for up to 80 hours. Control wells containing only BxPC-3 cells were monitored in parallel to provide baseline proliferation profiles. The xCELLigence system recorded real-time changes in cell index, which reflect alterations in tumor cell adhesion, proliferation, and viability in response to Vδ²⁺ γδ T-cell-mediated cytotoxicity.

Real-Time Monitoring and Data Acquisition: Impedance values were collected automatically at predefined intervals throughout the 80-hour monitoring period. The cell index signal increased steadily in control wells containing only BxPC-3 cells, reflecting normal tumor cell proliferation. In contrast, wells containing Vδ²⁺ γδ T cells displayed a rapid decline in cell index shortly after effector addition, indicating disruption of tumor cell adherence and viability. The magnitude and rate of decrease varied in accordance with the effector-to-target ratio, with higher Vδ²⁺ γδ T cell numbers producing more pronounced cytotoxic effects.

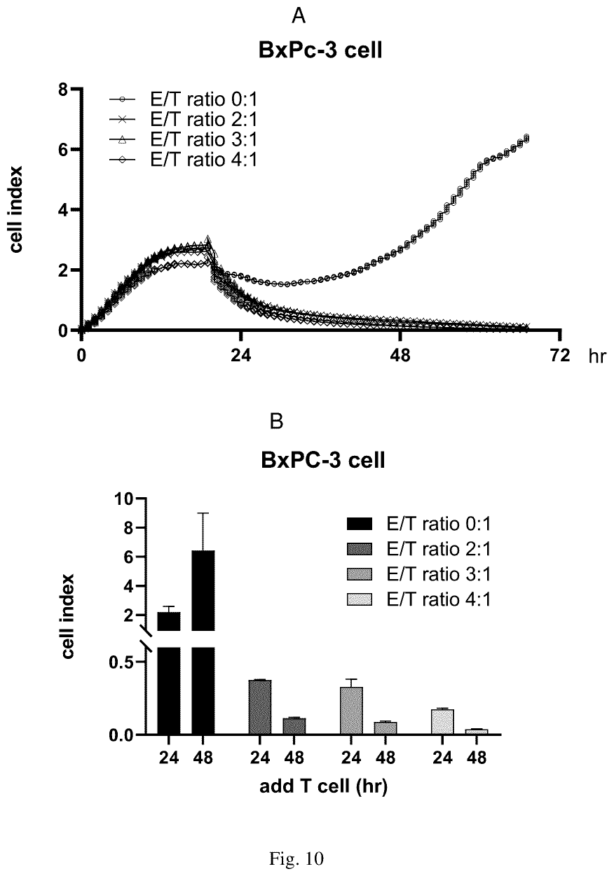

The impedance-based xCELLigence system continuously monitored cell growth over 80 hours. BxPC-3 cells cultured alone showed progressive proliferation, whereas co-culture with γδ T cells resulted in a rapid and sustained decrease in cell index, indicating potent cytotoxicity. Quantitative comparison of cell indices at 24 and 48 hours after γδ T cell addition confirmed significant growth inhibition in all treatment groups, with near-complete suppression of BxPC-3 cell proliferation at ratios of 1:4. These data demonstrate that unmodified γδ T cells exert strong, dose-dependent cytotoxic effects against pancreatic cancer cells in vitro (FIG. 10).

### Example 11 γδ T Cells Exhibit Superior Cytotoxic Effects Against GBM Cells (U87MG)

Preparation and Culture of GBM Cells: U87MG-luc human glioblastoma cells were obtained from ATCC and maintained in Dulbecco's Modified Eagle Medium supplemented with 10% fetal bovine serum and 100 μg/mL penicillin/streptomycin. Cells were cultured under standard incubation conditions until they reached logarithmic growth. Prior to co-culture experiments, U87MG-luc cells were seeded into 96-well plates at densities optimized for luciferase-based viability assays and were allowed to adhere overnight.

Preparation and Expansion of γδ T Cells and αβ T Cells: Peripheral blood mononuclear cells (PBMCs) were isolated from fresh human peripheral blood or leukopak collections obtained from healthy donors using Ficoll-Paque density-gradient centrifugation. After separation, PBMCs were washed twice with phosphate-buffered saline and resuspended in KBM581 medium supplemented with ten percent human platelet lysate. The cells were seeded at an initial density of 1×10⁶ cells per milliliter in tissue culture flasks. Zoledronic acid was prepared from a concentrated stock solution and added to the culture medium on Day 0 at a final concentration of 5 μM. Recombinant human IL-2 was added at 1000 IU/mL at the start of culture and replenished every two to three days thereafter. Cultures were maintained at 37° C. in a humidified incubator with 5% CO₂ for a total of 14 days, during which the cell density was kept between 0.5×10⁶ and 2×10⁶ cells per milliliter by periodic addition of fresh medium. At the end of the 14-day expansion period, the mixed T-cell population was subjected to TCRαβ depletion using a commercially available αβ T-cell depletion kit following the manufacturer's instructions. During magnetic separation, cells that bound to the αβ-specific microbeads remained attached within the column matrix and were subsequently collected as the αβ T-cell fraction. In contrast, cells that did not express TCRαβ passed freely through the column and were recovered as the γδ T-cell-enriched flow-through fraction. These γδ T cells were used for all downstream cytotoxicity and functional assays, while the retained αβ T cells served as the comparator population in the experiments.

Flow Cytometry Phenotyping of αβ and γδ T-Cell Populations: Flow cytometry was performed to distinguish αβ and γδ T-cell subsets within the CD³⁺/CD45⁺ T-cell population. Staining included TCRαβ antibodies, and representative plots demonstrated that αβ T cells contained predominantly TCRαβ⁺ cells, whereas γδ T cells formed a distinct TCRαβ⁻ population. This confirmation ensured that downstream cytotoxicity comparisons reflected true biological differences between the two T-cell types.

Co-Culture of T Cells with U87MG-luc GBM Cells: To assess cytotoxic activity, U87MG-luc cells were co-cultured with either γδ T cells or αβ T cells at tumor-to-effector ratios of 1:1 and 1:0.5. T cells were added directly to the wells containing U87MG-luc cells, and co-cultures were incubated for 24 hours under standard conditions. Control wells containing U87MG-luc cells alone were included to establish baseline viability and caspase activation levels.

Luciferase-Based Viability Measurement: At the end of the 24-hour co-culture period, U87MG-luc cell viability was assessed using luminescence-based quantification on the GloMax® Discover Multimode Reader, as described in the experimental documentation. Luminescence measurements were normalized to control wells to determine relative survival of GBM cells following exposure to γδ or αβ T cells. This assay enabled sensitive detection of GBM cell viability based on luciferase signal intensity reduction, which directly correlates with cytotoxicity.

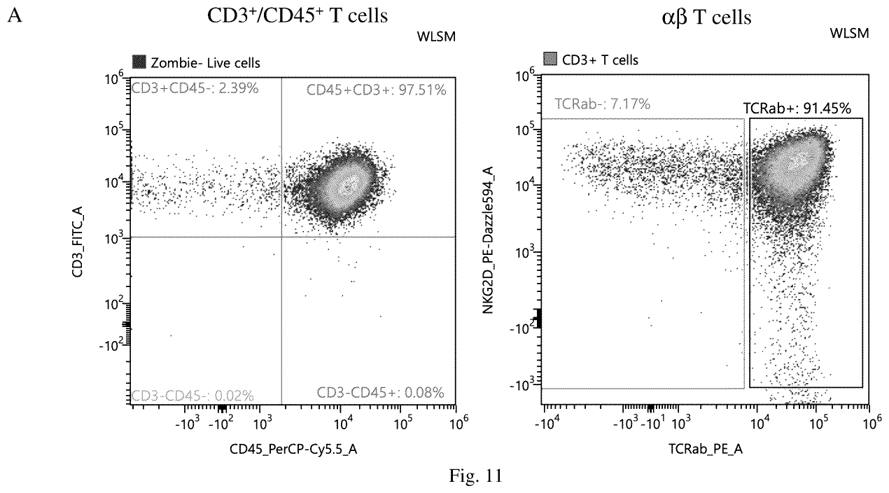

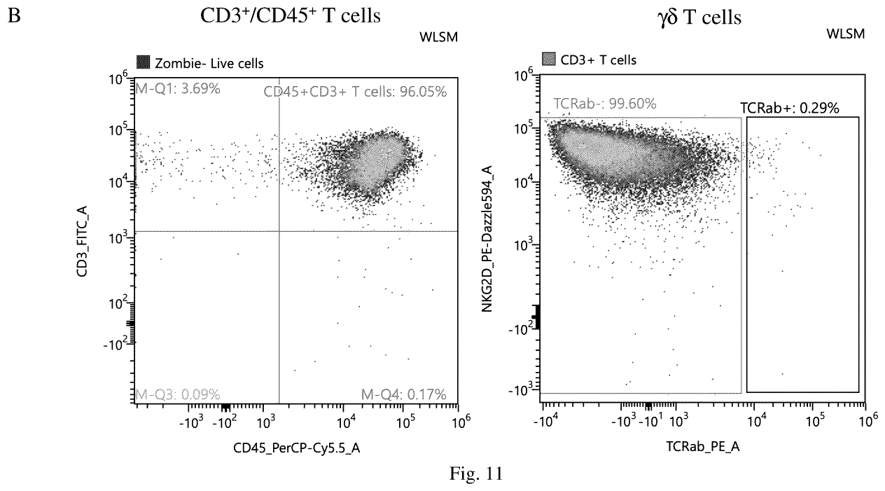

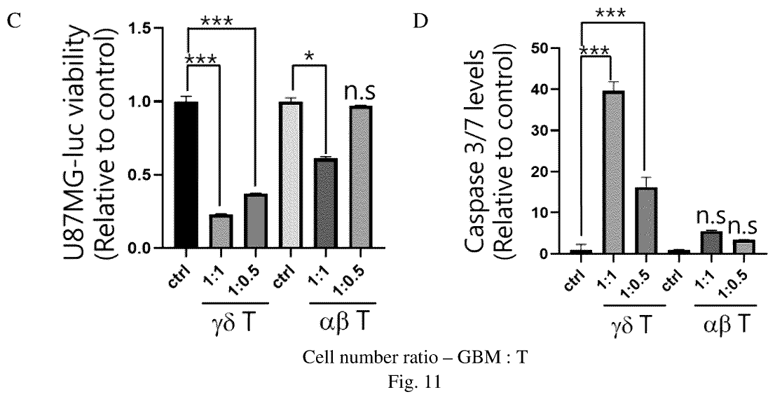

Caspase 3/7 Activity Assay for Cytotoxicity Assessment: To further characterize cell death mechanisms, supernatants were collected from co-cultures and mixed with reagents from the Glo-Caspase 3/7 Assay Kit based on the manufacturer's instructions. After incubation, caspase activity was measured using the GloMax® Discover platform. Increased caspase 3/7 levels indicated apoptosis induced by T-cell cytotoxic effects. Comparison between γδ T cell-treated and αβ T cell-treated groups provided additional mechanistic insight into differential killing efficiency (FIG. 11).

### Example 12 In Vivo Anti-Tumor Efficacy of Unmodified γδ T Cells in an Orthotopic Glioblastoma (U87MG) Mouse Model

Establishment of the Orthotopic Glioblastoma Model: An orthotopic glioblastoma model was generated using immunodeficient NOD.CB17-Prkdc^{scid}/NCrCrl mice. U87MG-luc human glioblastoma cells were maintained in DMEM supplemented with 10% fetal bovine serum and 100 μg/mL penicillin/streptomycin prior to implantation. Tumor implantation was performed stereotactically under anesthesia, with 1×10⁴ U87MG-luc cells delivered into the right striatum in a total injection volume of 5 μL. Following implantation, mice were monitored for recovery and tumor establishment.

Bioluminescence Imaging for Tumor Confirmation: To verify tumor engraftment, bioluminescence imaging was conducted on Day 0. Mice received an intraperitoneal injection of D-luciferin (15 mg/mL, 200 μL), followed by imaging on the IVIS Lumina III XRMS system (PerkinElmer) to quantify baseline tumor burden. Luminescent signal intensity was used as the reference point for subsequent response assessments.

Intracranial Administration of Vδ²⁺ γδ T Cells: Unmodified Vδ²⁺ γδ T cells were expanded ex vivo under serum-free conditions and prepared fresh on the day of administration. The Vδ²⁺ γδ T cells were delivered intracranially at the established tumor site immediately following baseline imaging on Day 0. The injection was performed using the same stereotactic coordinates employed for tumor implantation to ensure direct delivery into the tumor mass. The administered dose was selected based on prior optimization studies evaluating Vδ²⁺ γδ T-cell persistence and functional activity in the intracranial compartment.

Follow-Up Tumor Imaging and Assessment of Anti-Tumor Activity: To evaluate the early therapeutic effects of Vδ²⁺ γδ T-cell treatment, bioluminescence imaging was repeated on Day 2 using the same luciferin dosage and imaging parameters as baseline. A marked reduction in tumor-associated luminescence was observed relative to Day 0 measurements, indicating a substantial decrease in viable tumor cell burden within 48 hours of Vδ²⁺ γδ T-cell administration. The observed suppression of bioluminescent signal demonstrated rapid and robust anti-tumor activity mediated by intracranially delivered Vδ²⁺ γδ T cells.

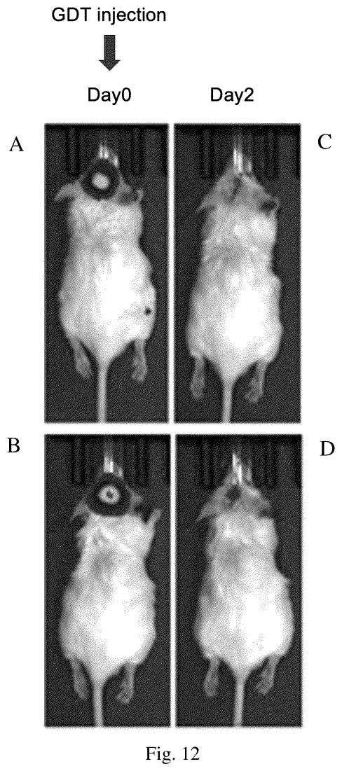

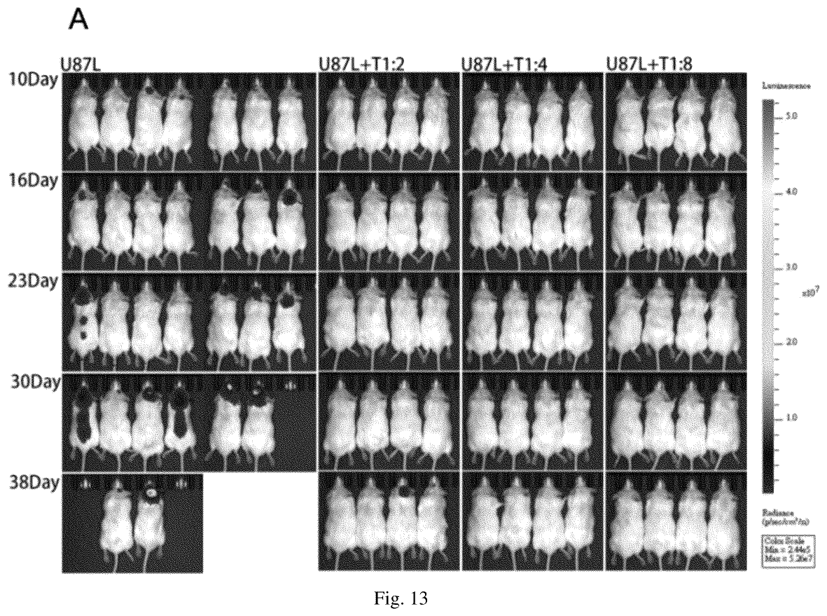

An orthotopic glioblastoma model was established by stereotactic implantation of U87MG-luc cells into the right striatum of immunodeficient mice. On Day 0, baseline bioluminescence imaging (BLI) was performed to confirm successful tumor engraftment. Unmodified γδ T cells (GDT) were administered intracranially at the tumor site, as indicated by the injection marker. Follow-up BLI on Day 2 demonstrated a marked reduction in luminescence intensity compared to baseline, indicating early anti-tumor activity of the administered GDT cells (FIG. 12).

### Example 13 Inhibition of Glioblastoma Growth by Co-Injection of Unmodified γδ T Cells in an Orthotopic U87MG Mouse Model

Establishment of the Orthotopic Glioblastoma Co-Injection Model: Human U87MG-luc glioblastoma cells were maintained in DMEM supplemented with 10% fetal bovine serum until use. For orthotopic implantation, mice were anesthetized and positioned in a stereotactic frame. Tumor cells were prepared at a concentration of 1×10⁴ cells in a 5-μL suspension. To evaluate the prophylactic anti-tumor effects of Vδ²⁺ γδ T cells, freshly expanded Vδ²⁺ γδ T cells were mixed directly with U87MG-luc cells immediately prior to implantation. The mixtures were prepared at U87MG:T-cell ratios of 1:2, 1:4, and 1:8. A total volume of 5 μL was delivered stereotactically into the right striatum using the same coordinates employed for U87MG-luc implantation alone. All mice were monitored during recovery and observed for signs of discomfort or neurological impairment.

Longitudinal Bioluminescence Imaging (BLI) for Tumor Monitoring: Tumor progression was evaluated non-invasively through serial bioluminescence imaging. Mice received intraperitoneal injections of D-luciferin (15 mg/mL, 200 μL) prior to imaging sessions. BLI was performed on Days 10, 16, 23, 30, and 38 post-implantation using the IVIS Lumina III XRMS system. Photon flux (radiance) measurements were captured under identical imaging conditions for all timepoints to ensure comparability. In the U87MG-luc-only cohort, robust and progressive increases in luminescence were observed, confirming rapid tumor expansion. In contrast, mice co-injected with Vδ²⁺ γδ T cells exhibited markedly reduced or completely absent luminescent signals, indicating inhibition of tumor formation.

Quantitative Analysis of Tumor Burden: Radiance values were quantified from acquired BLI images using Living Image analysis software. Mice in the U87MG-luc-only group demonstrated high photon flux values beginning at Day 10, with further escalation at later timepoints. A dose-dependent reduction in photon emission was observed in the Vδ²⁺ γδ T-cell co-injection groups, with the 1:4 and 1:8 groups showing no detectable signal throughout the entire study period. These findings confirmed that co-administration of Vδ²⁺ γδ T cells at the time of tumor inoculation effectively prevents tumor establishment in the orthotopic brain environment.

Survival Monitoring and Analysis: All mice were monitored daily for survival, weight loss, and neurological symptoms. Kaplan-Meier survival curves were generated to compare outcomes among treatment groups. Consistent with imaging results, mice in the U87MG-luc-only group exhibited progressive tumor-associated morbidity, resulting in significantly shorter survival. In contrast, survival was prolonged in all Vδ²⁺ γδ T-cell-treated groups, with the 1:4 and 1:8 groups exhibiting complete inhibition of tumor development and no tumor-related mortality during the study window. The survival benefit correlated directly with the degree of tumor growth suppression observed by BLI.

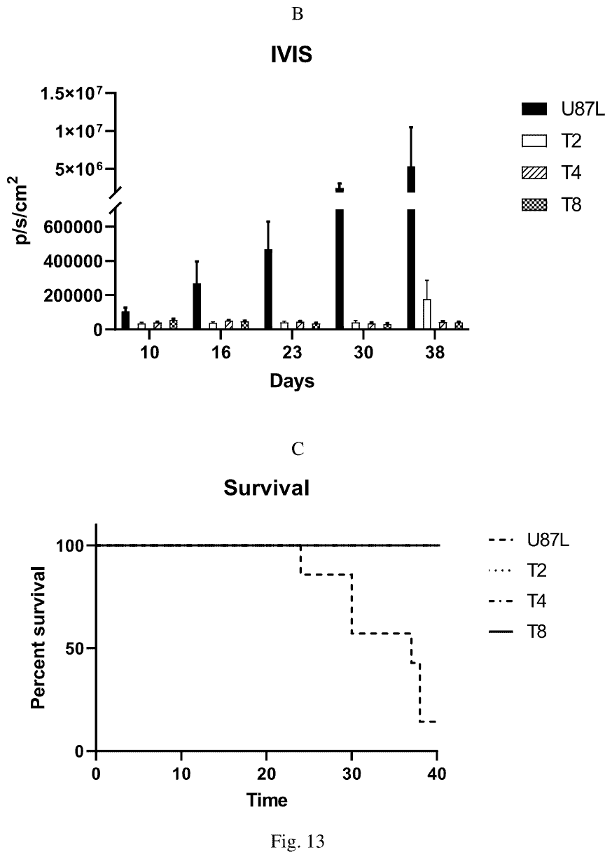

Images were acquired on Days 10, 16, 23, 30, and 38 after implantation. Robust tumor growth was observed in the U87L group, while co-injection with γδ T cells dose-dependently suppressed tumor formation. At E:T ratios of 1:4 and 1:8, no detectable bioluminescent signal was observed throughout the study period. These results indicate that co-administration of unmodified γδ T cells at the time of tumor cell inoculation effectively prevents glioblastoma establishment in vivo (FIG. 13).

What is claimed is:

1. A method for producing a γδ T cell population comprising enriched Vδ²⁺ γδ T cells, comprising:
(a) providing peripheral blood mononuclear cells (PBMCs); optionally the Vδ²⁺ γδ T cells contained in the PBMCs are separated by immunomagnetic separation to obtain a Vδ²⁺ γδ T cell subpopulation;
(b) culturing the PBMCs or Vδ²⁺ γδ T cell subpopulation in a medium containing zoledronic acid and one or more growth factors having interleukin-like activity to specifically enrich Vδ²⁺ γδ T cells to obtain an enriched Vδ²⁺ γδ T cell population; and
(c) harvesting the expanded Vδ²⁺ γδ T cell population.

2. The method according to claim 1, wherein after step (b), the method further comprises a step (e) of separating the enriched Vδ²⁺ γδ T cell population by immunomagnetic separation to remove non-Vδ²⁺ γδ T cells.

3. The method according to claim 1, wherein after step (b), the method further comprises a step (f) of expanding the enriched Vδ²⁺ γδ T cell population by culturing it for 7-28 days to obtain the expanded Vδ²⁺ γδ T cell population.

4. The method according to claim 1, wherein the PBMCs are obtained from a peripheral blood, umbilical cord blood, or fractions thereof.

5. The method according to claim 1, wherein the zoledronic acid is at a concentration of about 1 to about 30 μM.

6. The method according to claim 1, wherein the medium is free of serum.

7. The method according to claim 1, wherein the concentration of the growth factor having interleukin-like activity ranges from about 5 ng/ml to about 200 ng/ml or about 50 IU/mL to about 1,500 IU/mL.

8. The method according to claim 1, wherein the growth factor having interleukin-like activity is an interleukin.

9. The method according to claim 1, wherein the interleukin is IL-2, IL-4, IL-7, IL-15, IL-21, interleukin-1β or any combination thereof.

10. The method according to claim 1, wherein the interleukin is IL-2, IL-15, or a combination of IL-2 and IL-5.

11. The method according to claim 1, wherein the medium containing IL-15 maintains a constant expansion of γδ T-cells at a high level over time.

12. The method according to claim 1, wherein the zoledronic acid mediated γδ T-cell expansion can be increased by IL-15.

13. The method according to claim 1, further comprising synthetic growth factors and amino acids tailored for T cell expansion.

14. A cell population produced from a method according to claim 1.

15. The cell population according to claim 14, which comprises an enriched Vδ²⁺ γδ T cell subpopulation, wherein the enriched γδ T cell population comprises at least about 85%, 88%, 90%, 91%, 92%, 93%, 94%, 95%, 96%, 97%, 98% or 99% Vδ²⁺ T cells with over about 70%, 75%, 80%, 85%, 90%, 91%, 92%, 93%, 94%, 95%, 96%, 97%, 98% or 99% post-expansion viability.

16. The cell population of claim 14, wherein the γδ T cells are of therapeutic grade.

17. The cell population of claim 14, wherein the enriched γδ T cell population has low residual αβ T cells.

18. The cell population of claim 14, wherein the Vδ²⁺ γδ T cells express one or more CD3, CD45 and NKG2D in an intensity higher than about 70%, 75%, 80%, 85%, 90%, 95%, 96%, 97%, 98%, 98.5%, 99% or 99.5%.

19. A method for modulating an immune response, or treating a cancer or infectious disease in a subject, or vaccinating an animal, comprising administering the cell population of claim 16 to the subject.

20. The method according to claim 19, wherein the cancer is sarcoma, basal cell skin cancer, lymphoma, leukemia, lymphoproliferative disorder, plasmacytoma, histiocytoma, adenoma, carcinomas of solid tissues, hypoxic tumor, genitourinary cancer, hematopoietic cancer, nervous system cancer, bile duct cancer, cervical cancer, squamous cell cancer, endometrial cancer, esophageal cancer, head and neck cancer, glioblastoma (GBM) (such as glioblastoma multiforme), kidney cancer, hepatocellular carcinoma, liver cancer, lung cancer, pancreatic cancer, melanoma, Merkel cell cancer, mesothelioma, stomach cancer, breast cancer, or triple-negative breast cancer; and wherein the infectious disease is bacterial infections such as those caused by mycobacteria (e.g. tuberculosis), viral infections such as those caused by herpes simplex viruses (HSV), human immunodeficiency viruses (HIV), the hepatitis viruses, SARS-CoV-2 viruses, papillomaviruses (HPV), Epstein-Barr viruses (EBV), measles viruses, cytomegaloviruses (CMV), hepatitis C viruses (HCV), or influenza viruses, and parasitic infections such as those caused by plasmodium (e.g. malaria).
